(19) United States
(12) Patent Application Publication
(10) Pub. No.: US 2023/0108316 A1
(43) Pub. Date: Apr. 6, 2023

(54) METHODS FOR REHABILITATING HEART FAILURE USING GENE THERAPY

(71) Applicant: UNIVERSITY OF UTAH RESEARCH FOUNDATION, Salt Lake City, UT (US)

(72) Inventors: TingTing Hong, Salt Lake City, UT (US); Robin Mark Shaw, Salt Lake City, UT (US)

(21) Appl. No.: 17/801,002

(22) PCT Filed: Apr. 7, 2021
(86) PCT No.: PCT/US2021/026131

§ 371 Date: Aug. 19, 2022

Related U.S. Application Data
(60) Provisional application No. 63/088,123, filed on Oct. 6, 2020.
(60) Provisional application No. 63/007,229, filed on Apr. 8, 2020.

(51) Int. Cl. A61K 38/17; A61P 9/04

(52) U.S. Cl. CPC A61K 38/1719; A61P 9/04; A61K 48/00

(57) ABSTRACT

Described herein are compositions comprising viral vectors. The viral vectors may encode a t-tubule organizing protein or peptide such as cardiac isoform of bridging integrator 1 (cBIN1). Also disclosed herein are methods for treatment or prophylaxis of heart failure in a subject in need thereof. The method of treatment or prophylaxis may include administering a vector comprising cBIN1 to the subject for rehabilitating or increasing contractile (systolic) function or relaxation (diastolic) function in the heart of a subject having experienced heart failure or having chronic myocardial stress.

### CROSS-REFERENCE TO RELATED APPLICATIONS

This application claims priority to U.S. Provisional Patent Application N. 63/007,229, filed on Apr. 8, 2020, and 63/088,123, filed on Oct. 6, 2020, each of which is incorporated by reference herein in its entirety.

### FEDERALLY SPONSORED RESEARCH

This invention was made with United States government support under National Institutes of Health grant numbers HL133286, HL094414, and HL138577. The United States government has certain rights in the invention.

### TECHNICAL FIELD

Described herein are compositions comprising viral vectors. The viral vectors may encode a t-tubule organizing protein or peptide such as cardiac isoform of bridging integrator 1 (cBIN1). Also disclosed herein are methods for treatment or prophylaxis of heart failure in a subject in need thereof. The method of treatment or prophylaxis may include administering a vector comprising cBIN1 to the subject for rehabilitating or increasing contractile (systolic) function or relaxation (diastolic) function in the heart of a subject having experienced heart failure or having chronic myocardial stress.

### BACKGROUND

Heart failure (HF) is the fastest growing cardiovascular disorder affecting over 20 million people worldwide and 6.2 million Americans [1-2]. The majority of HF related mortality is associated with cardiac pump failure due to myocardial inotropic and lusitropic dysfunction, as well as sudden cardiac death due to increased arrhythmia burden of failing hearts. Furthermore, in nearly 50% of patients with HF with preserved ejection fraction (HFpEF) [2], severe diastolic failure with further increased arrhythmia risks occurs, which has even worse clinical outcomes and also lacks effective medical therapy. Thus, there is an urgent need to develop new therapeutic strategies that can limit and reverse heart failure progression.

During HF development, the pathophysiologic cellular hallmark of failing ventricular myocytes is abnormal calcium transients with impaired intracellular calcium homeostasis [3], which disrupts excitation-contraction (EC) coupling [4], impairs electrical stability [5], and disturbs mitochondrial metabolism [6]. Normal beat-to-beat calcium transient relies on a sequence of intracellular events known as calcium-induced-calcium-release (CICR) [7], where t-tubule L-type calcium channel (LTCC)-mediated initial calcium influx will subsequently induce a massive calcium release via ryanodine receptors (RyRs) from the sarcoplasmic reticulum (SR) store. During relaxation, the accumulated calcium will then be removed from the cytoplasm mainly by calcium reuptake to SR via SR Ca²⁺-ATPase (SERCA) together with calcium exclusion into the extracellular space [7]. In HF, abnormal t-tubule remodeling [8-10] impairs LTCC-RyR coupling and synchronous CICR [3, 11], resulting in diminished systolic release, EC uncoupling, and thus reduced contractility. On the other hand, HF-associated leaky RyRs [12] and abnormal SERCA2a function [13] will result in SR depletion and elevated diastolic calcium [14], resulting in severe diastolic failure and electrical instability [15]. In addition, impaired calcium homeostasis triggers loss of mitochondrial membrane potential [16] and increased permeability [17], which promotes the risk of mitochondrial-initiated cell death [18-19] and HF progression [18, 20]. Taken together, abnormal calcium homeostasis is critical in controlling normal cardiac pump function, electrical stability, and metabolism, which, when disturbed, will lead to pump failure, lethal arrhythmias, and severe metabolic disorder.

Cardiac transverse tubules (t-tubule) are critical for the initiation of calcium transients and maintenance of efficient excitation-contraction (EC) coupling. Pathological t-tubule remodeling is a consequence of β-adrenergic stimulation in HF [21-23]. Furthermore, impaired t-tubule microdomains have been implicated in HF progression [24-27]. In fact, t-tubule remodeling can be the tipping point from hypertrophy to failure [10]. Normal calcium transients [28], which require L-type calcium channels (LTCCs) to be at t-tubule microdomains, are crucial to cardiac contraction and relaxation. The t-tubule membrane scaffolding protein cardiac bridging integrator 1 (cBIN1) [29], which facilitates LTCC trafficking [30] and clustering for dyad organization, is also under the regulation of β-adrenergic receptor (β-AR) signaling [31]. Furthermore, cBIN1 is reduced in HF [31-33] and the resultant cBIN1-microdomain disruption impairs normal stress response, limiting contractility and promoting arrhythmias. Therapeutic approaches that preserve cBIN1-microdomains may benefit stressed hearts by protecting the calcium handling machinery, slowing HF progression.

Therefore, there remains a need for preventing remodeling within individual ventricular myocytes to improve overall cardiac remodeling and have therapeutic benefits for failing hearts.

### SUMMARY

One embodiment described herein is a method for rehabilitating heart tissue or ameliorating symptoms of heart failure in a subject having experienced heart failure or under chronic stress, the method comprising, diagnosing heart failure or myocardial stress in a subject; and administering a transgene encoding a Cardiac Bridging Integrator 1 (cBIN1) to heart tissue of the subject having experienced heart failure. In one aspect, the diagnosis of heart failure or myocardial stress comprises measuring reduced cBIN1 blood levels.

Another embodiment described herein is a method for rehabilitating or increasing contractile (systolic) function or relaxation (diastolic) function in the heart of a subject having experienced heart failure, the method comprising administering a transgene encoding Cardiac Bridging Integrator 1 (cBIN1) to heart tissue of the subject, wherein after the transgene is delivered to the heart tissue and expressed, contractile function of the heart is rehabilitated or increased. In one aspect, the transgene is administered after the subject is diagnosed with heart failure. In another aspect, the diagnosis of heart failure comprises measuring reduced cBIN1 blood levels. In another aspect, the method comprises administering the transgene to myocardium. In another aspect, the transgene is administered by injection. In another aspect, the transgene comprises a vector comprising the transgene encoding cBIN1. In another aspect, the transgene comprises about 1×10¹⁰ to about 5×10¹⁰ of vector genome. In another aspect, the expression of cBIN1 restructures damaged myocardium. In another aspect, the expression of cBIN1 stabilizes intracellular distribution of calcium handling machinery in the myocardium. In another aspect, the expression of cBIN1 reduces concentric hypertrophy in the myocardium. In another aspect, the expression of cBIN1 rehabilitates or increases t-tubule microfolds or microdomains in the myocardium. In another aspect, the expression of cBIN1 rehabilitates or decreases hyperphosphorylation of ryanodine receptor 2 (RyR2) in the myocardium. In another aspect, the expression of cBIN1 rehabilitates or improves cardiac contractility and lusitropy. In another aspect, the expression of cBIN1 rehabilitates or improves cardiac relaxation and diastolic function. In another aspect, the expression of cBIN1 is prophylactic for further damage to the myocardium. In another aspect, the transgene is administered at least once. In another aspect, the subject is mammal. In another aspect, the subject is a mouse or dog. In another aspect, the subject is a human. In another aspect, the subject experiences reduced ejection fraction (HFrEF).

Another embodiment described herein is the use of cBIN1 in a medicament for rehabilitation of myocardial tissue or repairing myocardial damage in a subject having experienced heart failure or having chronic myocardial stress.

Another embodiment described herein is the use of cBIN1 in a medicament for rehabilitating or increasing contractile (systolic) function or relaxation (diastolic) function in the heart of a subject having experienced heart failure or having chronic myocardial stress.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-B show the experimental protocol of cardiac bridging integrator 1 (cBIN1) post-treatment in mice subjected to transverse aortic constriction (TAC). FIG. 1A shows a schematic protocol: 47 mice were randomized into three groups: sham (N=12) or TAC mice with post-treatment of AAV9-GFP (N=17) and AAV9-cBIN1 (N=18) administered at 5 weeks post-TAC. FIG. 1B shows an echocardiography analysis of trans-aortic pressure gradient (TAP) in the three groups.

FIG. 2A-B show the post-treatment with exogenous cBIN1 improves survival rates in post-TAC mice. FIG. 2A shows Kaplan-Meier survival curves for all three groups of mice: sham controls (N=12), post-TAC mice treated with AAV9-CMV viruses transducing GFP (N=17) or cBIN1 (N=18). The log-rank test was used for comparison across three groups. FIG. 2B shows Kaplan-Meier survival curves for post-TAC mice not yet at end stage of disease prior to AAV9 injection (5-weeks post-TAC EF≥30%) following post-treatment of AAV9-GFP (N=16) or AAV9-cBIN1 (N=15). The log-rank test was used for comparison of survival rates between AAV9-GFP and AAV9-cBIN1 groups.

FIG. 3A-C shows that exogenous cBIN1 reduces TAC-induced hypertrophy and pulmonary edema. FIG. 3A shows longitudinal heart sections with H&E staining, (scale bar, 1 mm). FIG. 3B shows the ratio of heart weight over tibia length (HW/TL) and FIG. 3C shows lung weight over tibia length (LW/TL) at 20 weeks post-TAC. Data are presented as mean±SEM, and two-way ANOVA with Fisher's LSD test was used for statistical analysis. *,***p<0.05, 0.001 vs. sham; †p<0.05, comparing between GFP and cBIN1 groups.

FIG. 4A shows cBin1 gene transfer preserves myocardial systolic and diastolic function in pressure overloaded hearts. (A) Representative left ventricular (LV) short axis M-mode images from each group (sham, AAV9-GFP, AAV9-cBIN1) at 5 weeks post-TAC (pre-AAV9 injection) and 20 weeks post-TAC (15 weeks post-AAV9 injection). FIG. 4B-D show echocardiography-measured (FIG. 4B) left ventricle ejection fraction (EF), (FIG. 4C) end-diastolic volume, and (FIG. 4D) left ventricle mass at 5 (pre-AAV9) and 20 (post-AAV9) weeks post-TAC. FIG. 4E shows representative mitral valve inflow pulsed wave Doppler images (top) and tissue Doppler images of septal mitral valve annulus (e′) (bottom) at 20 weeks post TAC (15 weeks post-AAV9 injection). FIG. 4F shows quantification of E/e′ from each group at 5 (pre-AAV9) and 20 (post-AAV9) weeks post-TAC. FIG. 4G-H show delta changes from 5-week to 20-week in (FIG. 4G) stroke volume (SV) and (FIG. 4H) cardiac output (CO) of each mouse (ΔSV=SV_{20w}-SV_{5w}; ΔCO=CO_{20w}-CO_{5w}). Data are presented as mean±SEM, and two-way ANOVA with Fisher's LSD test was used for statistical analysis. *,**,***p<0.05, 0.01, 0.001 vs. sham; †,††,†††p<0.05, 0.01, 0.001 comparing between AAV9-GFP and -cBIN1 groups at each time point. ‡‡p<0.01 when comparing pre- and post-AAV9 treatment in each group.

FIG. 5A-C show AAV9-cBIN1 post-treatment rescues EF in post-TAC mouse hearts. FIG. 5A shows echocardiography monitored delta EF changes (ΔEF) from pre-AAV to 3, 6, 8, 10, and 15 weeks post AAV9 injection (correspondingly 8, 11, 13, 15, and 20 weeks post-TAC) in AAV9-CMV-GFP and -cBIN1 treating groups. FIG. 5B-C show histogram distribution of ΔEF at 6-week (FIG. 5B) and 8-week (FIG. 5C) post AAV9 treatment with Gaussian distribution fitting curve. Data are presented as mean±SEM.

FIG. 6A-F show that exogenous cBIN1 pretreatment improves myocardial pressure-volume (PV) loops. FIG. 6A shows schematic protocol: sham (N=5) or TAC with pretreatment of AAV9-GFP (N=10) or cBIN1 (N=10) administered 3 weeks prior to TAC. FIG. 6B-F show representative PV loop (FIG. 6B), EF (FIG. 6C), dp/dt max (FIG. 6D), dp/dt min (FIG. 6E) and Tau (FIG. 6F) in sham, AAV9-GFP and -cBIN1 hearts at 8 weeks post TAC surgery. Data are presented as mean±SEM, and one-way ANOVA with Fisher's LSD test was used for statistical analysis. **, *** p<0.01, 0.001 vs. sham; †, †† p<0.05, 0.01 comparing between AAV9-GFP and AAV9-cBIN1 groups.

FIG. 7A-C shows cBIN1-microdomain reduction in post-TAC hearts can be normalized with AAV9-cBIN1 pretreatment. FIG. 7A-B show a Western blot of (FIG. 7A) cBIN1, (FIG. 7B) ryanodine receptor (RyR), and Cav1.2 from sham, AAV9-GFP, and AAV9-cBIN1 pretreated post-TAC heart lysates. Quantitation is included in the bar graph at the bottom (n=8 hearts per group for cBIN1, n=6 hearts per group for cBIN1). FIG. 7C shows representative myocardial immunofluorescent spinning-disc confocal images of BIN1 labeling (anti-BAR domain; top panel), RyR (middle panel), and Cav1.2 (bottom panel) from sham, AAV9-GFP, and AAV9-cBIN1 pretreated post-TAC hearts. The insets include enlarged images of the corresponding boxes areas. Bottom row (from left to right): Peak power density of BIN1, RyR, and Cav1.2 distribution in sham, AAV9-GFP and cBIN1-pretreated hearts at 8 weeks post-TAC surgery (n=15-20 images from five hearts per group). Data are presented as mean±SEM, and one-way ANOVA with Fisher's LSD test was used for statistical analysis. *, **, *** p<0.05, 0.01, 0.001 vs. sham; †, †† p<0.05, 0.01 comparing between AAV9-GFP and -cBIN1 groups.

FIG. 8A-H show that exogenous cBIN1 reduces concentric hypertrophy in post isoproterenol (ISO) mouse hearts. FIG. 8A shows the experimental protocol: 56 mice were randomized into four experimental groups: AAV9-GFP+PBS, AAV9-GFP+ISO, AAV9-cBIN1+PBS, AAV9-cBIN1+ISO (n=14/group). FIG. 8B shows mouse heart weight to body weight ratio (HW/BW) in the four groups. FIG. 8C shows representative images of longitudinal axis view of left ventricles at both end diastolic and end systolic phase at 4 weeks post PBS or ISO infusion. FIG. 8D-G show echocardiography analysis of end diastolic volume, LV mass, and relative wall thickness (FIG. 8D), ejection fraction (FIG. 8E), E/e′ (FIG. 8F), stroke volume (FIG. 8G), and cardiac output (FIG. 8H) is also included. Data are presented as mean±SEM. Two-way ANOVA was used followed by Fisher's LSD test for multiple comparison. *, **, *** indicates p<0.05, 0.01, 0.001 for PBS vs. ISO comparison within each AAV9 treatment group; and ##, ### indicates p<0.01, 0.001 for GFP vs cBIN1 comparison within each drug infusion group.

FIG. 9A-C show that isoproterenol reduces cBIN1 and disrupts cBIN1-microfolds, which is normalized by AAV9-cBIN1. FIG. 9A shows Western blots of cBIN1 and GAPDH from heart lysates and immunoprecipitated heart lysates from GFP+PBS, GFP+ISO, cBIN1+PBS, and cBIN1+ISO hearts. Quantification in the bar graph to the right (N=6-7 hearts per group). FIG. 9B shows representative cardiomyocyte images with Di-8-ANNEPs labeling (top pane) (Scale bar, 10 μm) and power spectrum (bottom panel) of the corresponding boxed region of interest above. Quantification of peak power density is included in the bar graph to the left. (N=26-31 cells from 3-4 hearts per group). Data are presented as mean±SEM. Two-way ANOVA was used followed by Fisher's LSD test for multiple comparison. *, ** indicates p<0.05, 0.01 for PBS vs ISO comparison within each AAV9 treatment group; and #, ## indicates p<0.05, 0.01 for GFP vs cBIN1 comparison within each drug infusion group. FIG. 9C shows transmission electron microscopy imaging of t-tubule microfolds from myocardial tissue from all four groups (Scale bar, 1 μm). Quantitation of the degree of contour of t-tubules (TT) from each group is included in the bar graphs to the left (N=232-305 TTs from 60-100 images of 5-6 myocardial sections and 2-3 hearts from each group). Chi-square test was used to compare TT contour between groups, p<0.001 for comparison of GFP+PBS vs. GFP+ISO, GFP+ISO vs. cBIN1+ISO, and cBIN1+PBS vs other groups.

FIG. 10A-D show cBIN1 increases Cav1.2 localization to t-tubules. FIG. 10A shows a Western blot of Cav1.2 in heart lysates from GFP+PBS, GFP+ISO, cBIN1+PBS, and cBIN1+ISO hearts. Quantification (Cav1.2/GAPDH and Cav1.2/Troponin) is included in the bar graphs to the right (n=5-7 hearts per group). FIG. 10B shows representative confocal images (100×) of anti-Cav1.2 labeling in mouse myocardium from each group (top two panels) (Scale bar, 10 μm). The third panel includes power spectrum and the fourth panel includes fluorescence intensity profiles within the boxed areas along the cardiomyocyte longitudinal axis. FIG. 10C shows quantification of Cav1.2 peak power density and immunofluorescent intensities at t-tubules in each group (n=15-32 cell images from 3-4 hearts per group). Scale bar: 10 μm. FIG. 10D shows representative calcium transient tracing from each group and quantification of peak amplitudes (ΔF/F₀) (n=61-88 cells from 6 hearts per group). Data are presented as mean±SEM. Two-way ANOVA was used followed by Fisher's LSD test for multiple comparison. *** indicates p<0.001 for PBS vs ISO comparison within each AAV9 treatment group; and ### indicates p<0.001 for GFP vs cBIN1 comparison within each drug infusion group.

FIG. 11A-D shows cBIN1 organizes intracellular distribution of SERCA2a in post-isoproterenol hearts. FIG. 11A shows Western blot of SERCA2a in heart lysates from GFP+PBS, GFP+ISO, cBIN1+PBS, and cBIN1+ISO hearts. FIG. 11B shows quantification (SERCA2a/Actin) is included in the bar graph (n=6-8 hearts per group). FIG. 11C shows representative confocal images of anti-SERCA2a labeling in mouse myocardium from each group (top two panels). Scale bar: 10 μm. The third panel includes the power spectrum of SERCA2a of the boxed area above. FIG. 11D shows quantification of peak power density of SERCA2a (n=11-15 cell images from 3-4 hearts per group). Data are expressed as mean±SEM. Two-way ANOVA was used followed by Fisher's LSD test for multiple comparison. **, *** indicates p<0.01, 0.001 for PBS vs ISO comparison within each AAV9 treatment group; and ### indicates p<0.001 for GFP vs cBIN1 comparison within each drug infusion group.

FIG. 12A-B shows sucrose gradient fractionation of cardiac microsomes. FIG. 12A shows representative Western blots of Cav1.2 and cBIN1 in the F4 (TT) fraction of cardiac microsome from GFP+PBS, GFP+ISO, cBIN1+PBS, cBIN1+ISO hearts (2.5 μg protein loaded per lane). Quantification is included in the bar graphs (n=3 hearts per group). FIG. 12B shows representative Western blots of RyR, phospholamban (PLN), and SERCA2a in the F2 (longitudinal SR enriched) and F3 (jSR enriched) fractions of cardiac microsome from GFP+PBS, GFP+ISO, cBIN1+PBS, and cBIN1+ISO hearts (25 μg protein loaded per lane). Quantification of SERCA2a in F2 and F3 is included in the bar graphs to the right (n=3 hearts per group). Data are expressed as mean±SD. Two-way ANOVA was used followed by Fisher's LSD test for multiple comparison. *, **, *** indicates p<0.05, 0.01, 0.001 for PBS vs ISO comparison within each AAV9 treatment group; and #,## indicates p<0.05, 0.01 for GFP vs cBIN1 comparison within each drug infusion group.

FIG. 13A-D shows exogenous cBIN1 brings together Cav1.2-RyR and SERCA2a-cBIN1 molecules in cardiomyocytes. Super-resolution STORM imaging and nearest neighbor analysis of Cav1.2-RyR (FIG. 13A-B) and SERCA2a-cBIN1 (FIG. 13C-D) molecules in cardiomyocytes isolated from GFP+PBS, cBIN1+PBS, GFP+ISO, and cBIN1+ISO hearts. FIG. 13A, C show top-to-bottom, representative 2D-STORM cell images; representative 3D-STORM images of couplons; and histogram of nearest neighbor distance distribution obtained from full-cell 3D-STORM images. FIG. 13B, D show quantification of the first peak of nearest neighbor distance distribution histogram using full-cell image analysis (N=7-17 cells from 2-3 animals per group). Data are expressed as mean±SD. Two-way ANOVA was used followed by Fisher's LSD test for multiple comparison. * indicates p<0.05 for PBS vs ISO comparison within each AAV9 treatment group; and #, ### indicates p<0.05, 0.001 for GFP vs cBIN1 comparison within each drug infusion group.

FIG. 14A-H show echocardiography of post-isoproterenol hearts receiving AAV9-GFP, cBIN1, BIN1, BIN1+17, and BIN1+13. FIG. 14A shows representative LV short axis M-mode images from each group at baseline (top) and 4 weeks after isoproterenol treatment (bottom). In all the 4w post-ISO images, papillary muscles are marked by arrows. FIG. 14B-D show quantitative analysis of LV mass (FIG. 14B), relative wall thickness (FIG. 14C), and ejection fraction (FIG. 14D) from each group (N=10 mice per group). FIG. 14E shows representative mitral valve inflow pulsed wave Doppler images (top) and tissue Doppler images of septal mitral valve annulus 4-weeks after isoproterenol treatment (bottom). FIG. 14F-H show quantitative analysis of E/e′ (FIG. 14F), stroke volume (FIG. 14G), and cardiac output (FIG. 14H) from each group (N=10 mice per group). Data are expressed as mean±SEM. Two-way ANOVA was used followed by Fisher's LSD test for multiple comparison. *, **, *** indicates p<0.05, 0.01, 0.001 vs. Baseline; #, ##, ### indicates p<0.05, 0.01, 0.001 vs. the GFP group at 4w post-ISO.

FIG. 15A-J show cBIN1 gene transfer improves heart failure free survival in post-TAC mice. FIG. 15A shows schematic protocol for the TAC study. FIG. 15B shows trans-aortic pressure gradient measurement in all mice 5 days post-surgery. FIG. 15C shows Kaplan-Meier survival curves for heart free survival (non-survival is death or EF<35%) in WT vs. Bin1 HT mice (left), and AAV9-GFP or cBIN1 pretreated mice (right). Log-rank test was used for survival comparison. HW/BW (FIG. 15D) and LW/BW (FIG. 15E) in all mice at 8 weeks post-TAC. (FIG. 15F) Representative M-mode echocardiography images of all mice 8 weeks after surgery. Echocardiography-measured left ventricular ejection fraction (FIG. 15G), end diastolic volume (FIG. 15H), LV mass (FIG. 15I), and E/e′ (FIG. 15J) for all mice at 8 weeks post-TAC. Data are expressed as mean±SEM. Representative E and e′ images in the AAV9 treatment groups are included in the right panel of (FIG. 15J). Unpaired T-test (or nonparametric Mann-Whitney test used for comparison between WT and Bin1 HT. One-way ANOVA or Kruskal-Wallis test followed by Fisher's LSD test for multiple comparison was used for comparison among Sham, AAV9-GFP, and AAV9-cBIN1. *, **, *** indicates p<0.05, 0.01, 0.001 for comparison vs. WT or Sham; g, #⁴¹ indicates p<0.05, 0.01 for comparison of AAV9-GFP vs. AAV9-cBIN1.

FIG. 16 shows AAV9-transduced exogenous GFP-V5 and cBIN1-V5 protein expression in mouse cardiomyocytes. Representative adult mouse ventricular cardiomyocyte images under transmission light (top) or widefield fluorescent light (rabbit anti-V5 labeling, bottom) from control (left), AAV9-GFP-V5 (middle), or AAV9-cBIN1-V5 (right) treated mice.

FIG. 17 shows AAV9-transduced exogenous cBIN1 normalizes cardiomyocyte t-tubule microfolds in post-TAC hearts. Representative live-cell membrane labeling (Di-8-ANNEPs) images of cardiomyocytes freshly isolated from sham, AAv9-GFP and AAV9-cBIN1 treated post-TAC hearts. Quantification of t-tubule Di-8-ANNEPs intensity is included in the bar graph to the right (n=10 images from 5 hearts per group). All data are presented as mean±SEM. Kruskal-Wallis with LSD post-test was used for statistical analysis. ***, p<0.001 when comparing to Sham group; †, p<0.05 when comparing between AAV9-GFP and AAV9-cBIN1 groups.

FIG. 18A shows representative fluorescent confocal images (20×) of V5 and WGA labeling in myocardial cryosections obtained from mice 7 weeks after injection of AAV9 transducing GFP-V5 or cBIN1-V5 or control hearts without AAV9 injection (negative control). Positive V5 signal is detected in 63% and 57% of cells from hearts 7 weeks after retro-orbital injection of AAV9-GFP-V5 or AAV9-cBIN1-V5 (3×10¹⁰ vg), respectively. Scale bar, 100 μm. FIG. 18B shows quantitation of percent of myocardial area with detectable V5 signal. N=4-6 myocardial sections from 2-3 animals from each group. Data are presented as mean±SEM. Kruskal-Wallis test was used followed by Dunns's test for multiple comparison. * indicates p<0.05 for vs no AAV9 negative control.

FIG. 19A shows echocardiography-based categorization of LV remodeling in GFP+PBS, cBIN1+PBS, GFP+ISO, and cBIN1+ISO hearts. FIG. 19B shows representative Western blot and quantification of a-smooth muscle actin in hearts from each group. Data are presented as mean±SEM. Two-way ANOVA was used followed by Fisher's LSD test for multiple comparison. * indicates p<0.05 for PBS vs ISO comparison within each AAV9 treatment group; and ## indicates p<0.01 for GFP vs cBIN1 comparison within each drug infusion group.

FIG. 20A shows representative Western blot of total RyR2 protein expressions in mouse with quantifications. FIG. 20B shows representative confocal images (100×) of RyR2 in mouse cardiomyocytes from each group followed by power spectrum analysis (n=33-36 cells from 3-4 hearts per group). Scale bar: 10 μm.

FIG. 21 shows representative Western blots of RyR2 (total and phosphorylated pS2814 and pS2808), Cav1.2, CAMK116 (total and phosphorylated pT287), and phospholamban (PLN, total and phosphorylated pS16 and pT17). Quantifications are included in the bar graphs to the right. Data are presented as mean±SEM. N=4-7 hearts per group. Two-way ANOVA was used followed by Fisher's LSD test for multiple comparison. * indicates p<0.05 for PBS vs ISO comparison within each AAV9 treatment group; and # indicates p<0.05 for GFP vs cBIN1 comparison within each drug infusion group.

FIG. 22A shows a schematic protocol of sucrose gradient fractionation of cardiac microsomes (3-6 mg per heart). The yield of total amount protein recovered from each fraction F1, F2, F3, F4 is between 0.001-0.02, 0.4-0.8, 0.04-0.06, and<0.008 mg per heart prep, respectively. FIG. 22B shows representative Western blots of Cav1.2, Na⁺/K⁺-ATPase, SERCA2a, cBIN1, and caveolin 3 from the microsome input (M) and recovered fractions from F1, F2, F3, F4, and pellet from GFP+PBS, GFP+ISO, cBIN1+PBS, and cBIN1+ISO hearts.

FIG. 23A-E show cBIN1 organizes LTCC and SERCA2a in post isoproterenol mouse hearts. FIG. 23A shows representative Western blots of Cav1.2 and SERCA2a in post isoproterenol mouse hearts treated by AAV9-GFP, cBIN1, BIN1, BIN1+17, and BIN1+13. Quantification is included in the bar graphs to the right (n=3 hearts per group). FIG. 23B-C show representative confocal images (100×) of anti-Cav1.2 (FIG. 23B) and anti-SERCA2a (FIG. 23C) labeling in mouse myocardium from each group. Scale bar: 5 μm. FIG. 23D shows quantification of t-tubule Cav1.2 fluorescent intensity from each group. N=16-22 cells from 3 hearts from each group. FIG. 23E shows quantification of SERCA2a peak power density from each group. N=20 cell images from 2-3 hearts from each group. Data are expressed as mean±SEM. One-way ANOVA was used followed by Fisher's LSD test for multiple comparison. *, **, ***, indicate p<0.05, 0.01, 0.001 when compared to control GFP group.

FIG. 24A-D show that AAV9-cBIN1 rescues diabetic HFpEF in db/db mice. Echocardiography measured E/A (FIG. 24A), E/e′ (FIG. 24B), and SV (FIG. 24C); as well as (FIG. 24D) maximal running distance on a mouse treadmill in control db/m mice (db/m+GFP) and diabetic db/db mice treated with AAV9-GFP or cBIN1 (db/db+GFP or db/db+cBIN1). N=10 animals per group. One-way ANOVA followed by LSD fisher's exact test was used to compare differences across groups. *, *** indicate p<0.05 or 0.001 respectively when compared with db/m+GFP group. †, ††† indicate p<0.05 or 0.001 for comparison between db/db+GFP versus db/db+cBIN1 groups.

FIG. 25 shows that AAV9-cBIN1 rescues ischemic HFrEF in dogs. Echocardiography measured left ventricular ejection fraction (LVEF) versus week of study in two studied dogs (dog #1 and #2). Time 0 corresponds to time of LAD ligation. Arrows indicate the time of cBIN1 therapy.

### DETAILED DESCRIPTION

The reorganization of intracellular calcium handling machinery can be achieved by targeting t-tubule membrane microdomains organized by that cardiac isoform of bridging integrator 1 (cBIN1) [34]. It was previously found that cBIN1-microdomains organize LTCC-RyR dyads [12,14] by facilitating intracellular trafficking [13] and surface clustering of LTCCs [14, 35], affecting the electrochemical gradient across LTCCs via generating a protective slow diffusion zone within t-tubule lumen for extracellular ions [12], and recruiting RyRs to jSR for coupling with LTCCs [14]. More recently, it was found that cBIN1-microdomain is also critical in organizing the intracellular distribution of SERCA2a for diastolic calcium regulation [34]. In HF, cBIN1-microdomains are disrupted due to transcriptional reduction in cBIN1 [16, 36, 37], impairing dyad formation, calcium transient regulation, and cardiac contractility. Reduced myocardial cBIN1 can be detected in human blood, a result of cBIN1-membrane turnover and microparticle release [38]. In humans, plasma CS (cBIN1 score) is an index of myocyte cBIN1 level, which identifies myocardial structural remodeling, facilitating HF diagnosis and prognosis [39]. In mouse hearts subjected to chronic stress, pretreatment with exogenous cBIN1 preserves the microdomain-organized distribution of Cav1.2 and SERCA2a, maintaining normal inotropy and lusitropy. These data indicate that cBIN1 replacement can be an effective HF therapy with the potential to recover myocardial function in hearts with preexisting HF.

Since increased afterload is an important primary and secondary cause of HF [40], the current study uses a mouse model of elevated afterload induced by transverse-aortic constriction (TAC). In TAC mice, it was reported that cBIN1 pretreatment prevents HF development. Here we further used AAV9-mediated gene transfer to introduce exogenous cBIN1 in post-TAC mouse hearts with pre-existing HF. cBIN1 post-treatment reduces TAC-induced pathological remodeling, as well as the onset of HF and death. In mouse hearts with pre-existing TAC-induced HF, cBIN1 induces functional recovery. Also, in the present disclosure, we explored whether in vivo over-expression of exogenous cBIN1 can limit myocardial remodeling and dysfunction. Continuous isoproterenol infusion, which causes reduced myocardial cBIN1 expression and disorganized intracellular distribution of calcium handling proteins, also induces pathologic concentric hypertrophy with diastolic dysfunction. We found that normalization of cBIN1 through adeno-associated virus 9 (AAV9) mediated gene transfer both increases inotropy and preserves lusitropy, reducing pathologic hypertrophy. Within cardiomyocytes, we found that exogenous cBIN1 preserves the intracellular distribution of LTCCs at t-tubules, and the localization of the sarcoplasmic reticulum (SR) calcium-ATPase 2a (SERCA2a). The protective effects of cBIN1 are both isoform specific and confirmed effective in a second model of transverse aortic constriction (TAC) induced cardiac hypertrophy and HF, indicating that exogenous cBIN1 mediated preservation of t-tubule microdomains is a possible therapeutic approach to improve myocardial function in hearts under chronic stress.

AAV9 virus transduced-exogenous cBIN1 in myocardium, applied after a reduction in ejection fraction, can rescue cardiac systolic function and limit further development of ventricular chamber dilation and HF in mice subjected to chronic pressure overload stress.

Under continuous pressure overload, myocardial remodeling starts with an adaptive hypertrophic response followed by transitioning into maladaptive cardiac dilatation, leading to worsening HF [41-43]. In previous studies, we proved that the administration of AAV9-cBIN1 prior to TAC surgery preserves myocardial systolic and diastolic function, indicating the efficacy of cBin1 gene therapy in HF prevention. In the current disclosure, we found that exogenous cBIN1 administration not only limits but rescues the TAC-stressed hearts from further HF development and improves the overall survival with attenuated cardiac hypertrophy and lessened pulmonary edema in mice. Furthermore, exogenous cBIN1 introduced by gene transfer improves myocardial remodeling and cardiac function as measured by echocardiography. Most strikingly, mice with pre-existing severe HF exhibited recovered EF following cBin1 gene therapy, indicating the protective effect of exogenous cBIN1 may serve as a translatable treatment for patients with diagnosed pre-existing structural remodeling and HF.

Recently, AAV-mediated gene therapy has been shown as a promising modality for the treatment of HF [44-45]. There are currently several completed or ongoing clinical trials of HF gene therapies targeting various pathways such as the p-adrenergic system, Ca²⁺ cycling proteins, and cell death pathways, as well as homing stem cells [46]. We recently found that targeting the calcium regulating microdomains at t-tubules can be effectively achieved by transducing the essential microdomain-organizing protein cBIN1 [34]. By stabilizing t-tubule microdomains, cBIN1 potentially restores cytosolic calcium homeostasis and contributes to increasing systolic calcium release, improving diastolic reuptake, limiting SR leak for electrical stability maintenance, as well as preserving mitochondrial function to limit mitochondrial-associated cell death. The results indicate that this microdomain-targeting approach may serve as a new therapeutic strategy with improved efficiency in functional preservation, improving overall HF survival. Furthermore, the observed cBIN1-mediated improvement in overall survival is a possible combined effect from improved pump function and reduced arrhythmias, both of which are regulated by cBIN1-microdomains [7, 12, 36]. How cBIN1 therapy affects arrhythmia burden in failing hearts will need further analysis using in vivo telemetry monitoring in future studies. In addition, since TAC-induced HF is associated with mitochondrial disorder-associated myocyte death [47], it remains interesting in future studies to explore whether cBIN1 replacement therapy can preserve mitochondrial function and limit mitochondrial-related cell death in failing hearts.

With regard to functional recovery, although EF changes monitored from the beginning of AAV9-cBIN1 treatment shows a high peak of recovery at week 6 post-AAV9 followed by descending therapeutic efficiency, the rescue effect is maintained at 15-week post AAV9 injection. These data indicate even a single administration of AAV9-cBIN1 at a relatively low dose (3×10¹⁰ vg) is sufficient to preserve cardiac function. Whether multiple administrations of exogenous cBIN1 with increased dosage are needed to maximize its therapeutic effect remains to be tested. Nevertheless, our current rescue data indicate that, for patients with existing HF, cBin1 gene therapy could potentially break the worsening cycles of HF progression and result in functional recovery of failing hearts.

This study reveals a protective role of exogenous cBIN1 in mouse hearts with existing HF after subjected to pressure overload. For this first proof-of-concept study, we used the AAV9 vector driven by the CMV promoter for gene delivery due to its consistent transduction efficiency and established cardiac tropism. Further experiments using cBin1 packaged in AAV9 with a more efficient cardiac-specific promoter in mice and large mammals will be needed before clinical trials testing the efficacy and efficiency of cBin1 gene therapy in HF patients. Future studies are also needed to explore the intracellular mechanism for cBIN1 in balancing calcium homeostasis among cytosolic microdomains at t-tubules, SR, and nearby mitochondria. Further understanding of the downstream targeting molecules and signaling pathways of cBIN1 will be needed as well for a better understanding of the interplay between cBin1 gene therapy and HF pathophysiology.

This disclosure also indicates a beneficial effect of exogenous cBIN1 in preventing LV hypertrophy and cardiac dysfunction in stressed hearts. In mice subjected to continuous isoproterenol infusion, exogenous cBIN1 offers an isoform-specific improvement in cardiac inotropy and lusitropy, limiting the development of LV hypertrophy. The cardiac protective effect of exogenous cBIN1 is further confirmed in mouse hearts subjected to pressure overload induced HF.

Chronically elevated catecholamine levels and activation of cardiac β-adrenergic receptors (β-ARs) have a critical role in the pathogenesis of HF. Impaired myocardial structure and function have been observed in animals subjected to sustained sympathetic activation [48-49]. Isoproterenol, which is a synthetic catecholamine and non-selective β-AR agonist, has been used in research to induce the model of LV hypertrophy and dysfunction [50]. A high dose of isoproterenol was used here to induce LV concentric hypertrophy with preserved systolic function. Chronic excessive cardiac workload induced LV hypertrophy is associated with elevated risk of cardiovascular events [51] and preventing or reversing ventricular hypertrophy with preserved cardiac diastolic function is crucial to preventing the progression of stressed hearts to failing hearts. Here we found that cBIN1 attenuates chronic isoproterenol-induced hypertrophy and at the same time conveys an isoform-specific improvement in stroke volume and cardiac output in hypertrophic hearts with preserved systolic function. The increase of LV volume in the cBIN1 hearts is not secondary to pump failure and dilated cardiomyopathy, but rather it reflects improvement in myocardial lusitropy (E/e′) with a parallel increase of intrinsic myocardial contractility (inotropy). This phenotype of cBIN1 hearts is typical of athletic hearts in adaptation endurance training as characterized by chamber enlargement and increases of LV volume, stroke volume, and cardiac output [52-54]. Aerobic exercise training has been reported to improve myocardial function and inotropic and lusitropic responses in both animal models [55-56] and patients with hypertension [57] and diastolic failure [58]. Thus, exogenous cBIN1 may provide additional exercise like benefit to patients with heart failure, improving exercise capacity and quality of life.

These post-isoproterenol hearts are at a stage of hypertrophy with preserved systolic function, in which exogenous cBIN1 can effectively translate the increased demands on the heart into a functional effect. As a result, these functional and efficient cBIN1 hearts have limited hypertrophy development, which will likely prevent the next step of disease progression and HF development as occurs in the clinical setting. Next, the functional protective effect of exogenous cBIN1 in already decompensated hearts is also observed in a mouse model of TAC-induced hypertrophy and HF. Under pressure overload, compensated hypertrophy is an adaptive response. Over time, the adaptive response concedes to cardiac dilatation and the ensuing remodeling process becomes maladaptive, leading to worsening HF. We found that the fate of dilated cardiomyopathy development in pressure overload stressed hearts is causally determined by myocardial content of cBIN1 protein. Following pressure overload, less cardiac BIN1 in genetically deleted Bin1 HT-TAC hearts is associated with more severe dilated cardiomyopathy, whereas greater cBIN1 with gene transfer improves cardiac systolic and diastolic function, limits HF, and improves HF-free survival. It remains unclear whether exogenous cBIN1 reduces myocyte death, which also contributes to LV dilation in post-TAC hearts. Future studies will be necessary to explore the effect of cBIN1 on myocyte survival in stressed hearts. Nevertheless, our data indicate that exogenous cBIN1 not only limits hypertrophy development in stressed hearts but also prevents myocardial transition from hypertrophy to dilated cardiomyopathy and HF in TAC mice.

The mechanism of improvement in cardiac inotropic function by cBIN1 is linked to its known effect in organizing t-tubule microdomains required for dyad organization and efficient EC coupling. cBIN1 creates t-tubule microfolds to organize a slow diffusion zone trapping extracellular t-tubule lumen ions, attracts LTCCs forward trafficking to t-tubules [30], clusters LTCCs that are already delivered to cell surface [35], and recruits RyRs to couple with LTCCs at dyads [31]. Here we confirm in vivo that exogenous cBIN1, rather than any other BIN1 isoforms, increases Cav1.2 localization to t-tubules. These results support that preserved cBIN1-microdomain with organized LTCC distribution is responsible for the observed positive inotropic effect in sympathetically overdriven cBIN1 hearts. Whether cBIN1-microdomain regulates LTCC phosphorylation and its functional response to sympathetic stress including a well-established β-subunit-modulated Cav1.2 channel response [59-60] awaits future experimental explorations. Furthermore, RyR is critical to inotropy and hyper-phosphorylated leaky RyR plays a role in HF progression [14]. Consistent with previous reports in isoproterenol model and human HF [14, 61], we found that chronic isoproterenol activates PKA and CAMKII-induced RyR hyperphosphorylation. AAV9-cBIN1 blunts these pathways, normalizing RyR phosphorylation following chronic sympathetic activation and preventing SR leak.

An additional novel finding from the current disclosure is that exogenous cBIN1 increases SERCA2a function through organizing its intracellular distribution. Chronic isoproterenol-induced concentric hypertrophy with preserved systolic function is associated with disorganized intracellular distribution of SERCA2a yet increased overall protein expression. It is well accepted that SERCA2a activity is decreased in end stage HF. Our data indicate that in addition to reduced expression and impaired regulation by PLN, intracellular distribution of SERCA2a may also contribute to abnormal SR calcium reuptake activity in HF. Furthermore, as reported in adult rat ventricular cardiomyocytes with α-receptor agonist phenylephrine induced-hypertrophy, an adaptive increase in SERCA2a protein expression can occur due to elevated diastolic calcium-induced calcineurin/NFAT activation [62]. Thus, increased SERCA2a protein expression here is a possible adaptive response induced by elevated diastolic calcium concentration as indicated in elevated calcium-dependent phosphorylation at T287 of CAMKII. Thus, a transient increase in SERCA2a may occur at an early stage of all LV hypertrophy with preserved function. During disease progression, this adaptive increase in total SERCA2a protein expression will level off and even decrease as occurs in end stage HF, resulting in severe diastolic and systolic failure. In cBIN1 hearts, organized SERCA2a along SR indicates better calcium reuptake, therefore less diastolic calcium overload for hearts still at compensated stage. These results are consistent with a previous study in a rat model of HF which identified that increased BIN1 expression is associated with SERCA2a expression [63]. Future studies exploring cBIN1 regulation of diastolic calcium concentration and calcineurin/NTAT pathways will be needed to further understand its role in regulating SERCA2a expression and activity during disease progression. Note the effect on SERCA2a organization is not cBIN1-specific and can be partially induced by other BIN1 isoforms particularly BIN1+17. This is consistent with the partial in vivo protective effects from BIN1+17 on cardiac hypertrophy and diastolic function. Whether and how BIN1 isoforms cooperate to organize SERCA2a distribution in normal and diseased cardiomyocytes require further exploration in future studies. Furthermore, through regulation of calcium handling machineries at SR including SERCA2a distribution and RyR phosphorylation, cBIN1 may help maintain normal SR calcium load. As a limitation of the current study, future experiments are needed to quantify the effect of cBIN1 on SR calcium load, calcium release and reuptake kinetics, and arrhythmogenic spontaneous calcium release in chronically stressed hearts.

Nevertheless, the most robust protection of both inotropy and lusitropy in sympathetic overdriven hearts is only observed in the cBIN1 group, indicating possible further beneficial effect on lusitropy from cBIN1-dependent improvement in LTCC localization and dyad organization. With isoform-specific improvement in dyad organization, less orphaned leaky RyRs accumulate outside of dyads [31], limiting calcium leak from SR and decreasing cytosolic calcium concentration during diastolic phase. Together with the newly identified role on SERCA2a organization, our data indicate that a cBIN1-microdomain related regulation offers a unique benefit in protecting cardiac lusitropy in addition to its inotropy effect. On the other hand, cBIN1 overexpression may also suppress the pathological effects of isoproterenol stimulation by enhancing the control of β-AR signaling and the compartmentalization of secondary messengers and calcium handling channels and pumps. Thus, by stabilizing t-tubule microdomains to regulate all aspects of calcium handling, cBIN1 produces a positive feedforward mechanism for efficient intracellular beat-to-beat calcium cycling. In future studies it will be interesting to identify whether exogenous cBIN1 alters β-AR expression, intracellular distribution, and functional regulation following chronic sympathetic activation.

In conclusion, we found that over-expression of exogenous cBIN1 is protective in mouse hearts subjected to chronic β-AR activation induced concentric hypertrophy as well as pressure overload induced hypertrophy and HF. Future experiments in large mammals with common natural heart failure comorbidities such as hypertension and diabetes will be needed. Improving the viral infectivity in cardiomyocytes can additionally help limit or prevent isoproterenol-induced membrane disruption in all cardiomyocytes, increasing the protective effect on the entire heart. Further experiments using cBin1 packaged in AAV9 with an efficient and cardiac specific promoter to induce sufficient exogenous protein expression in all cardiomyocytes will be needed before clinical trials testing the efficacy and efficiency of cBin1 gene therapy. Future studies are needed to establish whether cBIN1 will impact systemic hemodynamics and blood pressure. Finally, future studies are needed to explore how cBIN1-microdomain regulates the organization of intracellular calcium handling machineries, EC coupling, SR calcium load and release, diastolic calcium concentration and its downstream calcium signaling pathways, the interplay between signaling pathways of pathologic and physiologic hypertrophic remodeling, as well as the molecular transition from compensated hypertrophy to decompensated cardiomyopathy.

One embodiment described herein is a method for rehabilitating heart tissue or ameliorating symptoms of heart failure in a subject having experienced heart failure or under chronic stress, the method comprising, diagnosing heart failure or myocardial stress in a subject; and administering a transgene encoding a Cardiac Bridging Integrator 1 (cBIN1) to heart tissue of the subject having experienced heart failure. In one aspect, the diagnosis of heart failure or myocardial stress comprises measuring reduced cBIN1 blood levels.

Another embodiment described herein is a method for rehabilitating or increasing contractile (systolic) function or relaxation (diastolic) function in the heart of a subject having experienced heart failure, the method comprising administering a transgene encoding Cardiac Bridging Integrator 1 (cBIN1) to heart tissue of the subject, wherein after the transgene is delivered to the heart tissue and expressed, contractile function of the heart is rehabilitated or increased. In one aspect, the transgene is administered after the subject is diagnosed with heart failure. In another aspect, the diagnosis of heart failure comprises measuring reduced cBIN1 blood levels. In another aspect, the method comprises administering the transgene to myocardium. In another aspect, the transgene is administered by injection. In another aspect, the transgene comprises a vector comprising the transgene encoding cBIN1. In another aspect, the transgene comprises about 1×10¹⁰ to about 5×10¹⁰ of vector genome. In another aspect, the expression of cBIN1 restructures damaged myocardium. In another aspect, the expression of cBIN1 stabilizes intracellular distribution of calcium handling machinery in the myocardium. In another aspect, the expression of cBIN1 reduces concentric hypertrophy in the myocardium. In another aspect, the expression of cBIN1 rehabilitates or increases t-tubule microfolds or microdomains in the myocardium. In another aspect, the expression of cBIN1 rehabilitates or decreases hyperphosphorylation of ryanodine receptor 2 (RyR2) in the myocardium. In another aspect, the expression of cBIN1 rehabilitates or improves cardiac contractility and lusitropy. In another aspect, the expression of cBIN1 rehabilitates or improves cardiac relaxation and diastolic function. In another aspect, the expression of cBIN1 is prophylactic for further damage to the myocardium. In another aspect, the transgene is administered at least once. In another aspect, the subject is mammal. In another aspect, the subject is a mouse or dog. In another aspect, the subject is a human. In another aspect, the subject experiences reduced ejection fraction (HFrEF).

Another embodiment described herein is the use of cBIN1 in a medicament for rehabilitation of myocardial tissue or repairing myocardial damage in a subject having experienced heart failure or having chronic myocardial stress.

Another embodiment described herein is the use of cBIN1 in a medicament for rehabilitating or increasing contractile (systolic) function or relaxation (diastolic) function in the heart of a subject having experienced heart failure or having chronic myocardial stress.

It will be apparent to one of ordinary skill in the relevant art that suitable modifications and adaptations to the compositions, formulations, methods, processes, apparata, assemblies, and applications described herein can be made without departing from the scope of any embodiments or aspects thereof. The compositions, apparata, assemblies, and methods provided are exemplary and are not intended to limit the scope of any of the disclosed embodiments. All the various embodiments, aspects, and options disclosed herein can be combined in any variations or iterations. The scope of the compositions, formulations, methods, apparata, assemblies, and processes described herein include all actual or potential combinations of embodiments, aspects, options, examples, and preferences described herein. The compositions, formulations, apparata, assemblies, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed.

Should the meaning of any terms in any of the patents or publications incorporated by reference conflict with the meaning of the terms used in this disclosure, the meanings of the terms or phrases in this disclosure are controlling. All patents and publications cited herein are incorporated by reference herein for the specific teachings thereof.

Various embodiments and aspects of the inventions described herein are summarized by the following clauses:
Clause 1. A method for rehabilitating heart tissue or ameliorating symptoms of heart failure in a subject having experienced heart failure or under chronic stress, the method comprising, diagnosing heart failure or myocardial stress in a subject; and administering a transgene encoding a Cardiac Bridging Integrator 1 (cBIN1) to heart tissue of the subject having experienced heart failure.
Clause 2. The method of clause 1, wherein the diagnosis of heart failure or myocardial stress comprises measuring reduced cBIN1 blood levels.
Clause 3. A method for rehabilitating or increasing contractile function in the heart of a subject having experienced heart failure, the method comprising administering a transgene encoding Cardiac Bridging Integrator 1 (cBIN1) to heart tissue of the subject, wherein after the transgene is delivered to the heart tissue and expressed, contractile function of the heart is rehabilitated or increased.
Clause 4. The method of clause 3 wherein the transgene is administered after the subject is diagnosed with heart failure.
Clause 5. The method of clause 4, wherein the diagnosis of heart failure comprises measuring reduced cBIN1 blood levels.
Clause 6. The method of any one of clauses 1-5, wherein the method comprises administering the transgene to myocardium.
Clause 7. The method of any one of clauses 1-6, wherein the transgene is administered by injection.
Clause 8. The method of any one of clauses 1-7, wherein the transgene comprises a vector comprising the transgene encoding cBIN1.
Clause 9. The method of any one of clauses 1-8, wherein the transgene comprises about 1×10¹⁰ to about 5×10¹⁰ of vector genome.
Clause 10. The method of any one of clauses 1-9, wherein expression of cBIN1 restructures damaged myocardium.
Clause 11. The method of any one of clauses 1-10, wherein expression of cBIN1 stabilizes intracellular distribution of calcium handling machinery in the myocardium.
Clause 12. The method of any one of clauses 1-11, wherein expression of cBIN1 reduces concentric hypertrophy in the myocardium.
Clause 13. The method of any one of clauses 1-12, wherein expression of cBIN1 rehabilitates or increases t-tubule microfolds or microdomains in the myocardium.
Clause 14. The method of any one of clauses 1-13, wherein expression of cBIN1 rehabilitates or decreases hyperphosphorylation of ryanodine receptor 2 (RyR2) in the myocardium.
Clause 15. The method of any one of clauses 1-14, wherein expression of cBIN1 rehabilitates or improves cardiac contractility and lusitropy.
Clause 16. The method of any one of clauses 1-15, wherein expression of cBIN1 rehabilitates or improves cardiac relaxation and diastolic function.
Clause 17. The method of any one of clauses 1-16, wherein expression of cBIN1 is prophylactic for further damage to the myocardium.
Clause 18. The method of any one of clauses 1-17, wherein the transgene is administered at least once.
Clause 19. The method of any one of clauses 1-18, wherein the subject is mammal.
Clause 20. The method of any one of clauses 1-19, wherein the subject is a mouse or dog.
Clause 21. The method of any one of clauses 1-20, wherein the subject is a human.
Clause 22. The method of any one of clauses 1-21, wherein the subject experiences reduced ejection fraction (HFrEF).
Clause 23. Use of cBIN1 in a medicament for rehabilitation of myocardial tissue or repairing myocardial damage in a subject having experienced heart failure or having chronic myocardial stress.
Clause 24. Use of cBIN1 in a medicament for rehabilitating or increasing contractile (systolic) function or relaxation (diastolic) function in the heart of a subject having experienced heart failure or having chronic myocardial stress.

### EXAMPLES

### Example 1

Materials and Methods

Animal procedures for functional rescue studies. Adult male C57BL/6 mice (The Jackson Laboratory) were used. All mice were anesthetized at the age of 8-10 weeks and subjected to open-chest sham or transverse aortic constriction (TAC) surgery. TAC was performed by tying a 7-0 silk suture against a 27-gauge needle between the first and second branch of the aortic arch. For sham controls, age-matched mice were subjected to open-chest mock surgery without TAC being performed. For gene therapy, at 5 weeks post the onset of TAC, mice received retro-orbital injection of 100 μL of 3×10¹⁰ vector genome (vg) of AAV9 virus (Welgen, Inc.) transducing cBIN1-V5 or GFP-V5 [64].

Animal procedures for isoproterenol studies. For the isoproterenol study, adult male C57BL/6 mice were administered 3×10¹⁰ vector genome (vg) of AAV9 transducing GFP or BIN1 isoforms (Welgen, Inc.) via retro-orbital injection [64]. Three weeks after vg administration, mice were implanted subcutaneously with osmotic mini pumps releasing PBS or isoproterenol (30 mg/kg/day). 56 mice were randomized into GFP+PBS, GFP+ISO, cBIN1+PBS, or cBIN1+ISO group (N=14/group). Another 50 mice were randomized into receiving AAV9-GFP, cBIN1, BIN1, BIN1+17, or BI N1+13 (N=10/group) before isoproterenol. AAV9 was used since it is a promising gene therapy vehicle and exhibits the highest cardiac tropism [65]. The CMV promoter was used given its efficiency and safety in cardiac gene transfer [66]. AAV9-CMV-GFP was used as the negative control virus since it does not induce cardiomyocyte toxicity and has been successfully used as a negative control virus in numerous gene therapy studies with animal models of cardiovascular diseases [67]. For TAC study, either adult male cardiac-specific Bin1 heterozygotes (Bin1 HT; Bin1^{flox/+}, Myh6-cre⁺) with their wild type (WT; Bin1^{flox/+}, Myh6-cre⁻) littermates [29]; or adult male C57BL/6 mice (Jackson Laboratory) were used. All mice were anesthetized at the age of 8-10 weeks and subjected to open-chest TAC or mock surgery (Sham). For gene therapy, same as the isoproterenol study, mice received retro-orbital injection of 3×10¹⁰ vg of AAV9 virus transducing cBIN1-V5 or GFP-V5 at 3 weeks prior to the onset of TAC.

Isoproterenol mini pump study. Fifty-six mice were randomized to receive a dose of 3×10¹⁰ vector genome (vg) of AAV9 transducing V5-tagged GFP or cBIN1 via retro-orbital injection while mice were anesthetized with 1% isoflurane in oxygen [68]. Three weeks after viral injection, mice were subjected to implantation of osmotic mini pump releasing isoproterenol or PBS (N=14 per group for each of the four study groups: AAV9-GFP+PBS, AAV9-GFP+ISO, AAV9-cBIN1+PBS, AAV9-cBIN1+ISO). AAV9 was used in this study since AAV is the most promising gene therapy vehicle [21, 69] and AAV9 exhibits the highest cardiac tropism in mice (4-6). The CMV promoter was used since it has been established that AAV9-CMV can efficiently and safely direct cardiac gene transfer [25]. AAV9-CMV-GFP was used as the negative control virus since AAV9-CMVGFP does not induce cardiac damage and cardiomyocyte toxicity [25-26], and GFP AAV9 has been successfully used as a negative control virus in numerous gene therapy studies with animal models of cardiovascular diseases, including mouse models of hypertrophy and cardiomyopathy [26-29]. This protocol was also repeated in a second set of animals. Similarly, three weeks before isoproterenol mini pump implantation, fifty mice were randomized to receive a dose of 3×10¹⁰ vector genome (vg) of AAV9 transducing V5-tagged GFP, BIN1, BIN1+13, BI N1+17, or cBIN1 (n=10 per group) via retro-orbital injection. Three weeks after AAV9 injection, mice were implanted with subcutaneous ALZET osmotic minipump (Model 1004, Durect, Cupertino, Calif., USA) continuously releasing isoproterenol following previously established procedure [30]. In brief, under light anesthesia with inhalation of isoflurane, mice were implanted subcutaneously on the back with osmotic mini pumps, which continuously release isoproterenol at 30 mg/kg/day.

Transverse aortic constriction (TAC) study. For cBIN1 deficiency study, TAC was performed on male mice with cardiac-specific Bin1 heterozygote deletion (Bin1 HT; Bin1^{flox/+}, Myh6-Cre⁺) and their wild type (WT; Bin1^{flox/+}, Myh6-Cre⁻) littermates (WT) at the age of 8-10 weeks old. Bin1 HT and WT mice were generated as previously described. Specifically, heterozygote loxp site flanked Bin1 (loxP sites around exon 3 of the Bin1 gene) mice were interbred with Myh6-cre⁺ mice to generate cardiomyocyte specific Bin1 HT (N=10) and WT littermate controls (n=14). Genotypes were confirmed by PCR to differentiate Bin1⁺, Bin1^{flox}, and Cre⁺ alleles according to a previously established method. For AAV9 mediated over-expression study, 5 to 7-week old male C57BL/6J mice (Jackson Laboratory) received retro-orbital injection of AAV9 virus (3×10¹⁰ vg) transducing cBIN1-V5 (N=18) or GFP-V5 (N=18). After three weeks, mice were anesthetized at the age of 8-10 weeks and subjected to open-chest TAC surgery. Age-matched mice subjected to open-chest mock surgery without TAC being performed were used as sham controls (N=10). TAC was performed to induce pressure overload as previously described. Briefly, 8-12 weeks old male mice were anesthetized by face mask administration of 3% isoflurane and then intubated and placed on a ventilator (Harvard Apparatus) with supplemental O₂ and 1.5% isoflurane using a tidal volume of 0.2 mL and a respiratory rate of 120 breaths/min. The chest cavity was entered in the second intercostal space at the upper sternal border through a small incision, and aortic constriction was performed by tying a 7-0 nylon suture ligature against a 27-gauge needle between the first and second branch off the aortic arch. Subcutaneous buprenorphine (0.8 mg/kg) was administered for pain relief, and mice were allowed to recover in a heated chamber with 100% O₂. Animals were euthanized, and tissues harvested for analysis after 8 weeks of TAC.

Generation and administration of adeno-associated virus 9 (AAV9). All five AAV9 vectors expressing GFP-V5, BIN1-V5, BIN1+13-V5, BIN1+17-V5, and cBIN1-V5 (BIN1+13+17-V5) driven by the CMV promoter were custom made and produced at Welgen, Inc. (Worcester, Mass., USA). We used previously reported gateway expression clones of V5-tagged GFP and mouse BIN1 isoforms [31], which were sequenced and then sent to Welgen for subsequent cloning into AAV vector and viral preparation. Next, these gene inserts (GFP-V5 or BIN1-V5) were subcloned into the pAAV-CMV vector (Welgen, Inc., Worcester, Mass., USA), and the positive clones were selected by restriction enzyme digestion. The pAAV-CMV-(GFP/BIN1)-V5 plasmid DNA were purified and sequenced. All AAV viruses were produced in HEK293 cells. Three plasmids, pAAV-CMV-(GFP/BIN1)-V5, pAAV-rep/cap9, and pHelper vectors were transfected into 293 cells using polyethylenimine. Following transfection, the supernatant and cells were harvested. The AAV viruses were released from HEK293 cells by 3 freeze-thaw cycles. The viruses in the medium were precipitated using PEG8000 (Sigma-Aldrich, St. Louis, MO, USA). The cell lysate and pelleted supernatant precipitate were combined and treated by Benzonase (Merck, Kenilworth, N.J., USA) at 37° C. for 1 h. The virus was purified by iodixanol gradient centrifugation and concentrated with Amicon Ultra-15 centrifugal filter (Sigma-Aldrich, St. Louis, Mo., USA).

Echocardiography for functional rescue studies. In vivo systolic and diastolic left ventricular (LV) functions were monitored by echocardiography in anesthetized mice using Vevo 7700 at baseline, pre-surgery, and every other week thereafter until the end of the experimental protocol. The trans-aortic pressure gradient was recorded using the modified Bernoulli equation (ΔPressure gradient (mm Hg)=4×peak velocity² (m/s)²) at 2-weeks post-surgery. All surviving mice at 5 weeks post-TAC were included in the study.

Echocardiography for isoproterenol studies. Echocardiography were recorded using a Vevo-3100 ultrasound system (Visual Sonics) equipped with 70 MHz transducer. Protein interaction was analyzed by immunofluorescent imaging and biochemical coimmunoprecipitation. Peak intensity of Cav1.2 at t-tubules was quantified by Image J as previously reported [30]. Power spectrum analysis was analyzed in Matlab using FFT conversion [10, 30]. Intracellular protein distribution was analyzed by sucrose gradient fractionation using a previously established method [70]. For calcium transient measurement, Cal-520-AM (AAT Bioquest) was used as previously described [31]. Three-dimensional super-resolution stochastic optical reconstruction microscopy (STORM) images were obtained [31] for nearest neighbor analysis between LTCC-RyR and SERCA2a-cBIN1 molecules.

Primary endpoint of severe HF-free survival versus non-survival, and HF classification. Overall survival was analyzed in all groups. Furthermore, severe heart failure (HF)-free survival was also analyzed and compared between the AAV9-GFP and AAV9-cBIN1 groups. For severe HF-free survival, the primary endpoint is survival with ejection fraction (EF)≥35% measured by echocardiography. Non-survival is either death or EF<35% within 20-weeks post-TAC. At the end of the protocol, survived TAC mice were measured for tibial length (TL), lung weight (LW), and heart weight (HW).

Immunofluorescence Labeling and Confocal Imaging. For cardiomyocyte membrane fluorescent labeling, freshly isolated ventricular cardiomyocytes from GFP-TAC and cBIN1-TAC mice were incubated with Di-8-ANNEPs for 20 min at room temperature (RT). The cells were then washed with HBSS to remove the remaining dye before live-cell imaging. For fixed-cell V5 imaging (10×), isolated cardiomyocytes were fixed in methanol at −20° C. for 5 min and permeabilized and blocked with 0.5% Triton X-100 and 5% normal goat serum (NGS) in PBS for 1 h at RT. Cells were incubated with rabbit anti-V5 (Sigma) overnight at 4° C. and detected by Alexa555 conjugated goat anti rabbit IgG. For tissue immunofluorescent imaging, myocardial cryo-sections were fixed with ice-cold acetone for 5 min. The primary antibodies used were mouse anti-BIN1-BAR (2F11, Rockland), mouse anti-RyR (Abcam), or rabbit anti-Cav1.2 (Alomone). Following incubation with primary antibodies and several washes with 1×PBS, cells and tissue sections were then incubated with Alexa488 or Alexa555 conjugated goat anti-mouse or rabbit secondary antibodies (Life Technologies) and mounted with DAPI containing ProLong gold. All confocal imaging was performed on a Nikon Eclipse Ti microscope with a 100×1.49 numerical aperture (NA) and 60×1.1 or 10×objectives. High-resolution cardiomyocyte images were obtained using a spinning-disc confocal unit (Yokogawa CSU10) with diode-pumped solid state (DPSS) lasers (486 nm, 561 nm, 647 nm) generated from laser merge module 5 (Spectral applied research, Calif.). T-tubule membrane labeling fluorescent intensity profiles were generated by ImageJ, and peak intensity at t-tubules is quantified as previously reported [29]. Power spectrum analysis was analyzed in Matlab using FFT conversion and normalized peak power density at t-tubules was compared across groups [10, 30].

Immunofluorescence labeling and imaging with spinning disc confocal microscopy. Myocardial tissue sections were embedded in 100% OCT media and flash frozen on dry ice with ethanol and stored in a −80° C. freezer before being sectioned at 10 μm as previously reported [32]. After fixation by acetone, tissue cryosections were permeabilized with 0.1% Triton X-100 and 5% normal goat serum (NGS, Life Technology) in PBS for 1 h at RT. For V5, CaV1.2, and SERCA2a staining, tissue sections were incubated with primary antibodies against rabbit anti-V5 (1:500, Sigma-Aldrich, St. Louis, Mo., USA), rabbit anti-CaV1.2 (1:250, Alomone Labs, Jerusalem, Israel), or mouse anti-SERCA2a (1:250, Abcam, Cambridge, Mass., USA) overnight at 4° C. After several washes with 1×PBS, tissue sections were then incubated with goat anti-mouse and anti-rabbit IgG conjugated with Alexa 4#88 and 555, respectively. Tissue sections were mounted with DAPI containing Prolong® Gold medium. All imaging was obtained with a Nikon Eclipse Ti microscope with a 40×1.1 or 100×1.49 numerical aperture total internal reflection fluorescence objective and NIS Elements software (Nikon, Los Angeles, Calif., USA). Confocal Z stacks at Z-step increments of 0.5 μm were collected with a spinning-disk confocal unit (Yokogawa CSU10, Sugar Land, Tex., USA) connected to the same Ti microscope with diode-pumped solid state lasers (486 nm, 561 nm) generated from laser merge module 5 (Spectral Applied Research, Richmond Hill, Ontario, Canada), and captured by a high-resolution ORCA-Flash 4.0 digital CMOS camera. T-tubule Cav1.2 fluorescent intensity profiles were generated by ImageJ and peak intensity at t-tubules is quantified as previously reported [30]. Calcium transients were performed following previously described protocol [31]. Briefly, freshly isolated cardiomyocytes were loaded with 10 μmol/L Cal-520-AM (AAT Bioquest) in 0.4% Pluronic F-127 in normal Tyrode buffer for 30 minutes. After 3 washes in buffer containing 1 mmol/L probenecid cells were placed in imaging chamber and paced with field stimulator (lonflux) at 1 Hz. Images were collected using spinning disc confocal microscope at 67 fps and analyzed using Nikon Element Software. Fluorescent signals of F₀ (baseline fluorescence) and Fmax (maximal fluorescence at the peak of calcium transient) were background corrected first followed by ratio calculation of ΔF/F₀=(Fₘₐₓ-F₀)/F₀ for comparison across groups.

Power Spectrum Analysis. The frequency domain power spectrum of cardiomyocyte immunofluorescent subsections was generated in Matlab using FFT conversion [10, 30]. Power spectrum normalized to maximal component was generated and plotted over distance (1/frequency, μm). Normalized peak power density [71] was quantified and compared among groups.

Super-resolution Stochastic Optical Reconstruction Microscopy (STORM) Imaging and Nearest Neighbor Analysis. For STORM imaging, cardiomyocytes were prepared as previously reported [31]. On the day of imaging, fresh STORM imaging buffer (0.5 mg/mL glucose oxidase, 40 μg/mL catalase, and 10% glucose with mercaptoethylamine) was added to the dish. The STORM images were collected with the Nikon Eclipse Ti microscope with lasers (488 nm, 561 nm from a self-contained 4-line laser module with acousto-optic tunable filters) and captured by a high-speed iXon DU897 Ultra EMCCD camera. The STORM module was used to obtained and analyze the images to generate 3-dimensional (3D) projections of Cav1.2/RyR and cBIN1/SERCA2a images at nanoscale resolution. For nearest neighbor analysis, the native 3D STORM images are displayed with the gaussian rendering algorithm available in Nikon Elements software, and 3D stacks of 3D STORM images (two channels per acquisition, either Cav1.2/RyR or cBIN1/SERCA2a) in molecule list text file format were obtained at a z-spacing of 10 nm for a depth of 500 nm. The molecule list text files were imported in ImageJ and the nearest distance between molecules from two channels (nearest neighbor distance) was calculated. The nearest neighbor distances were constructed and displayed in user-defined range and bin-width as frequency distribution histogram and fitted in 15^{th}-degree polynomial curve with the first peak value detected. The distance between Cav1.2-RyR and SERCA2a-cBIN1 molecules at the corresponding first peak position were quantified and compared among groups.

Transmission Electron microscopy. All transmission electron microscopy (TEM) work was done by the core facility at the Electron Imaging Center of The California NanoSystems Institute, UCLA. Tissue preparation was performed using a previously reported method [72]. Briefly, mouse hearts were perfused with 20 mL of fresh fixative solution (2% glutaraldehyde and 2% paraformaldehyde in 1×PBS). Left ventricular tissue (1 mm³) were post-fixed with 1% osmium tetroxide and incubated in 3% uranyl acetate. After dehydration in ethanol, samples were treated with propylene oxide, embedded in Spurr resin (Electron Microscopy Services), and sectioned using an ultramicrotome (Leica). The sections were mounted on grid and stained with uranyl acetate and lead citrate before image acquisition using the JEM1200-EX, JEOL microscope (Gatan). The degree of contoured t-tubules was quantified using a modified scoring system established previously [29].

Western Blotting for functional rescue studies. Tissue lysates were made from hearts flash frozen in liquid nitrogen. Frozen tissue was homogenized in radio-immunoprecipitation assay (RIPA) lysis buffer as previously described [41]. Lysates were rotated head-to-toe in 4° C. for 40 min, sonicated, followed by centrifugation (16,000×g for 25 min at 4 ° C.) to clear cellular debris. Protein lysates were then prepared 2×sample buffer (Bio-Rad, Hercules, Calif.) containing 5% 8-mercaptoethanol, incubated in RT for 30 min, and separated on an 8-12% gradient sodium dodecyl sulfate (SDS) polyacrylamide electrophoresis gel. Proteins were electro-transferred to polyvinylidene difluoride (PVDF) membrane. After transfer, membranes were fixed in methanol and blocked with 5% BSA in 1×Tris-buffered saline (TBS) for 1 h at RT, and incubated with primary antibody in 5% BSA in 1×TBS overnight at 4° C., followed by incubation with Alexa 647 conjugated secondary antibody (Life Technology) for 1 h at RT. Primary antibodies consisted of a custom-made polyclonal rabbit anti-BIN1 exon 13 (Anaspec) [29], mouse anti-RyR (Abcam), rabbit Cav1.2 antibody (Alomone), and mouse anti-GAPDH (Millipore).

Western Blotting for isoproterenol studies. Frozen heart tissues were homogenized using RIPA lysis buffer with protease inhibitor, and a Bradford assay was used to determine the protein concentration. Samples were separated on NuPAGE™ Novex™ 4-12% Bis-Tris Protein Gels and then transferred to polyvinylidene difluoride membranes. After blocking for 1 h with 5% Bovine Serum Albumin (BSA) in 1×TNT buffer, membranes were incubated overnight at 4° C. with primary antibody including rabbit anti GAPDH or Actin (Sigma-Aldrich, St. Louis, Mo., USA), rabbit anti-CaV1.2 (Alomone Labs, Jerusalem, Israel), or mouse anti-SERCA2a (Abcam, Cambridge, MA, USA), followed by incubation with secondary antibody (goat anti rabbit or mouse IgG-Alexa 647) for 1.5 hour at room temperature (RT). Immunoreactive bands were imaged with the Molecular Imager® Gel Doc™ XR+System (Bio-Rad Laboratories, Irvine, Calif., USA) and band intensities quantified with Image Lab software (Bio-Rad Laboratories, Irvine, Calif., USA).

Cardiac microsome preparation and sucrose gradient fractionation. Microsome sucrose gradient fractionation was prepared according to an established protocol with modifications [70]. Myocardial membrane microsomes were prepared from starting material of one heart for each experimental group. Frozen heart tissue was homogenized with a Polytron Handheld homogenizer in 2 mL homogenization buffer (20 mM Tris pH 7.4, 250 mM sucrose, 1 mM EDTA supplemented with HALT protease inhibitor). The homogenate was then centrifuged at 12,000×g (Beckman) for 20 minutes at 4° C. and the supernatant (S1) was collected in a pre-weighed tube and kept on ice. The pellet was resuspended in 1 mL of the same buffer, homogenized, and centrifuged at 12,000×g for 20 minutes at 4° C. The supernatant (S2) was collected and combined with the S1 from previous step. The combined microsomal supernatant (S1+S2) was then subjected to ultracentrifugation at 110,000×g for 2 hours at 4° C. After ultracentrifugation, the supernatant was disposed, the pellet was weighted, and the appropriate amount of buffer (˜1 mL) was added to bring a final concentration of microsome ˜25 mg/mL. The total protein concentration in the resuspended microsome was measured using Nanodrop 2000 for each sample and normalized among the four groups. The same amount (3-6 mg in 0.5 mL) of total microsome from each sample was carefully laid over the top of a discontinuous sucrose gradient [52, 58, 73], and 45%, v/w in homogenization buffer, 2 mL each) and ultracentrifuged in a fixed angle MLA-55 rotor at 150,000×g for 16 hours with a Beckman Coulter Optima Max XP Benchtop Ultracentrifuge. Samples were then collected from the following fractions: F1, 27%; F2, 27/32%; F3, 32/38%; and F4, 38/45%; as well as the pellet (P) from the bottom of the tube. For each fraction, ˜1 mL was collected, diluted 4×in homogenization buffer and ultracentrifuged at 120,000×g for 2 hours at 4° C. The pellet was resuspended in 100 μL of homogenization buffer, followed by protein concentration measurement by Nanodrop 2000. The yield of total amount of protein recovered from each fraction F1, F2, F3, F4 is between 0.001-0.02, 0.4-0.8, 0.04-0.06, and <0.008 mg per heart, respectively. Sample buffer was added before samples were frozen and stored at −20° C. before subsequent Western Blot analysis.

Statistical analysis. All data are expressed as mean±standard error of the mean (SEM) or standard deviation (SD) as specified. Normality was assessed using the Shapiro-Wilk test. Kaplan-Meier survival analysis was used to compare across two groups using the log-rank test and across three groups using the log-rank trend test. Continuous variables were compared using T-test/Mann-Whitney U and one-way analysis of variance (ANOVA)/Kruskal-Wallis tests. Two-way ANOVA was used to determine differences between two groups at two different time points. Two-way ANOVA was used to determine differences between two AAV9 groups with different drug infusion, which was then followed by Fisher's least significant difference (LSD) post-hoc adjustment for multiple pairwise comparisons. Categorical variables were analyzed using Fisher's exact or Chi-square tests. Data were analyzed using GraphPad Prism (version 7.0; GraphPad Software, La Jolla, Calif., USA). Two-sided p values were used and p<0.05 is considered statistically significant.

### Example 2

Functional Rescue by Exogenous cBIN1 in Mouse Hearts with Pressure Overload-Induced Heart Failure

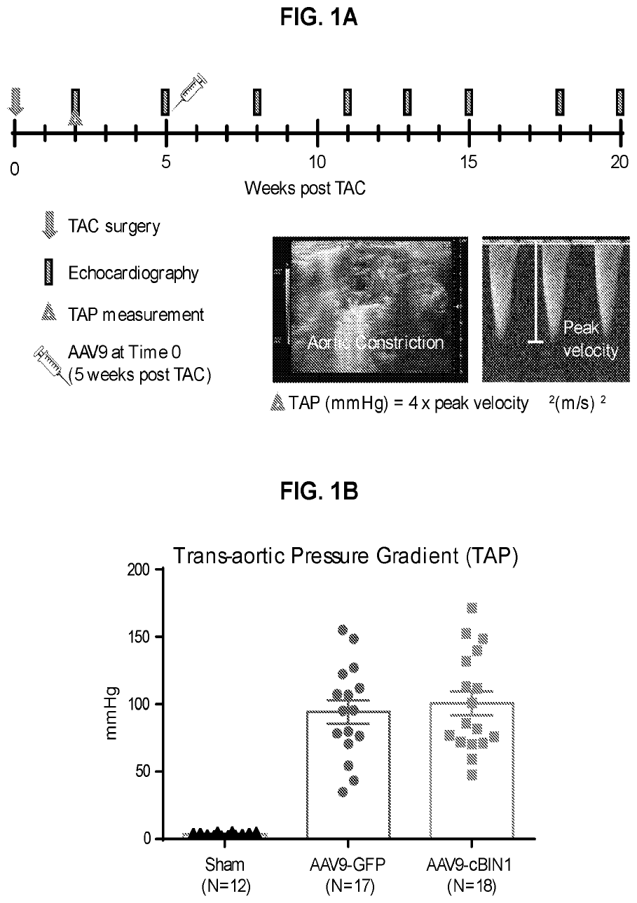

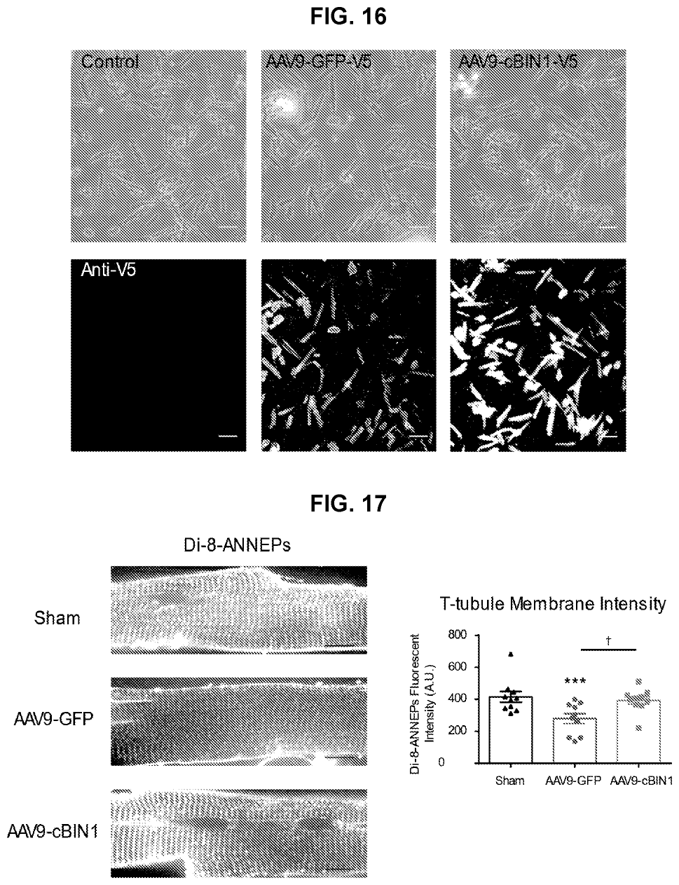

To explore whether targeting cBIN1-microdomain can be a new therapy for HF, how cardiac cBIN1 affects HF development in mice subjected to pressure overload stress was investigated. Either transverse aortic constriction (TAC) or a mock surgery (sham) was performed in adult male mice at the age of 8-10 weeks, followed by echocardiography monitoring to determine overall survival as well as severe systolic HF free survival (non-survival as death or ejection fraction, EF<35%). As indicated in the experimental protocol in FIG. 1A, mice were subjected to TAC first for 5 weeks before retro-orbital injection of AAV9 transducing cBIN1-V5 or control GFP-V5, followed by echocardiography monitoring for an additional 15 weeks after virus injection (20 weeks post-TAC). In addition to a group of mice subjected to an open-chest mock surgery (sham control, N=10), 36 mice were subjected to TAC surgery. One mouse died before reaching Time 0 (5 weeks post-TAC), the remaining 35 surviving mice were randomized to receive AAV9-GFP (N=17) or cBIN1 (N=18) at 3×10¹⁰ vg. Anti-V5 labeling of cardiomyocytes isolated from mice after 15 weeks of AAV9 injection identified positive V5 signal, indicating successful transduction of exogenous protein in cardiomyocytes (FIG. 16). Comparable trans-aortic pressure gradient at 2 weeks post-TAC (FIG. 1B) and myocardial dysfunction at 5 weeks post-TAC were observed in these mice as evidenced by a reduction in left ventricular (LV) EF with an increase in LV end diastolic volume (EDV) (Table 1) before AAV9 injection.

TABLE 1
Echocardiographic and physiological parameters of AAV9-GFP
or AAV9-cBIN1 injected mice before and after AAV9 injection

| | Pre-AAV9 (5 w-post TAC) | | | Post-AAV9 (20 w-post TAC) | | |
|---|---|---|---|---|---|---|
| | SHAM | GFP | CBIN1 | SHAM | GFP | CBIN1 |
| EF (%) | 53.72 ± | 41.87 ± | 44.22 ± | 48.25 ± | 27.65 ± | 41.72 ± |
| | 2.01 | 3.20** | 2.83* | 2.22 | 4.19*** | 1.72^{##} |
| LVEDV (μL) | 62.34 ± | 82.35 ± | 78.48 ± | 58.24 ± | 107.64 ± | 77.96 ± |
| | 3.87 | 5.32* | 6.22 | 2.52 | 10.01*** | 4.55^{##} |
| LVESV (μL) | 28.97 ± | 49.44 ± | 46.43 ± | 35.29 ± | 79.92 ± | 45.76 ± |
| | 2.46 | 4.96* | 5.89* | 2.03 | 10.43*** | 3.43^{###} |
| HR (bpm) | 482.04 ± | 516.60 ± | 481.41 ± | 463.77 ± | 557.35 ± | 504.94 ± |
| | 14.76 | 8.49 | 12.40 | 12.28 | 20.71*** | 8.91*, ^{##} |
| SV (μL) | 33.38 ± | 32.91 ± | 32.05 ± | 32.95 ± | 27.72 ± | 32.20 ± |
| | 2.17 | 2.38 | 1.19 | 1.98 | 3.28 | 1.79 |
| CO (ml/min) | 16.00 ± | 16.92 ± | 15.30 ± | 15.35 ± | 15.37 ± | 16.34 ± |
| | 1.08 | 1.18 | 0.53 | 1.17 | 1.83 | 1.08 |
| LVAWs (mm) | 1.40 ± | 1.61 ± | 1.56 ± | 1.35 ± | 1.52 ± | 1.61 ± |
| | 0.04 | 0.07* | 0.05 | 0.08 | 0.08 | 0.06* |
| LVAWd (mm) | 0.99 ± | 1.26 ± | 1.20 ± | 0.97 ± | 1.29 ± | 1.28 ± |
| | 0.03 | 0.06*** | 0.03** | 0.06 | 0.07*** | 0.05*** |
| LVPWs (mm) | 1.15± | 1.46 ± | 1.34 ± | 1.10± | 1.41 ± | 1.33 ± |
| | 0.07 | 0.07** | 0.06 | 0.03 | 0.15* | 0.07 |
| LVPWd (mm) | 0.79 ± | 1.15 ± | 1.06 ± | 0.84 ± | 1.24 ± | 1.11 ± |
| | 0.05 | 0.07*** | 0.04** | 0.03 | 0.16*** | 0.06* |
| LV Mass (mm) | 124.39 ± | 232.36 ± | 202.30 ± | 137.17 ± | 300.76 ± | 223.09 ± |
| | 4.76 | 15.20*** | 8.43*** | 9.60 | 29.55*** | 12.41***, ^{###} |
| BW (g) | 27.83 ± | 29.66 ± | 29.28 ± | 32.41 ± | 33.97 ± | 34.77 ± |
| | 0.65 | 0.56 | 0.40 | 1.19 | 0.84 | 0.95* |
| HW (mg) | — | — | — | 196.98 ± | 288.63 ± | 234.94 ± |
| | | | | 7.25 | 31.63*** | 10.75^{#} |
| LW (mg) | — | — | — | 158.32 ± | 245.70 ± | 165.28 ± |
| | | | | 5.14 | 63.18*** | 3.68^{#} |
| HW/TL (g/m) | — | — | — | 9.85 ± | 14.77 ± | 11.75 ± |
| | | | | 0.36 | 1.40*** | 0.54^{#} |
| LW/TL (g/m) | — | — | — | 7.92 ± | 12.53 ± | 8.26 ± |
| | | | | 0.26 | 3.09* | 0.18 |

EF, ejection fraction; LVEDV, left ventricular end-diastolic volume; LVESV, left ventricular end-systolic volume; HR, heart rate; SV, stroke volume; CO, cardiac output; LVAWs/LVAWd, left ventricular anterior wall in systole/diastole; LVPWs/d, left ventricular posterior wall in systole/diastole; LV Mass, left ventricular mass; BW, body weight; HW, heart weight; LW, lung weight; TL, tibia length.
Data are expressed as mean ± SEM.
*Indicates p < 0.05.
**Indicates p < 0.01.
***Indicates p < 0.001 vs. sham.
^{#}Indicates p < 0.05.
^{##}Indicates p < 0.01.
^{###}Indicates p < 0.001 for AAV9-GFP vs. AAV9-cBIN1.

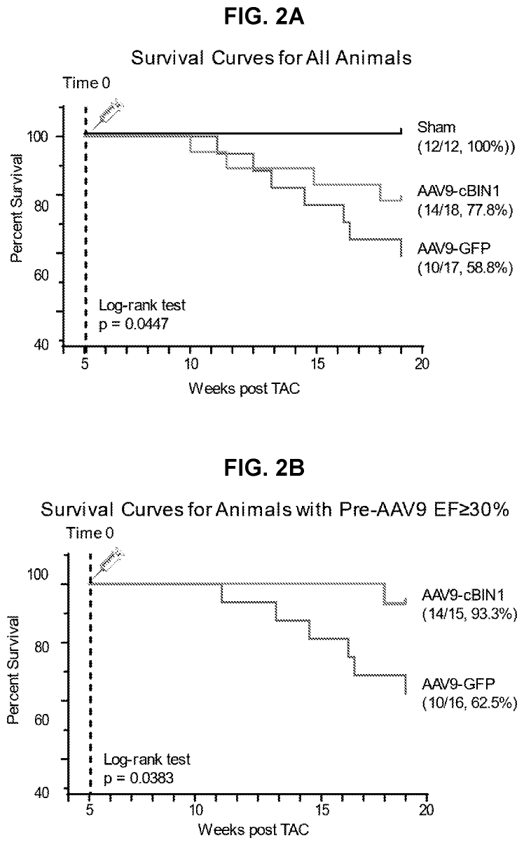

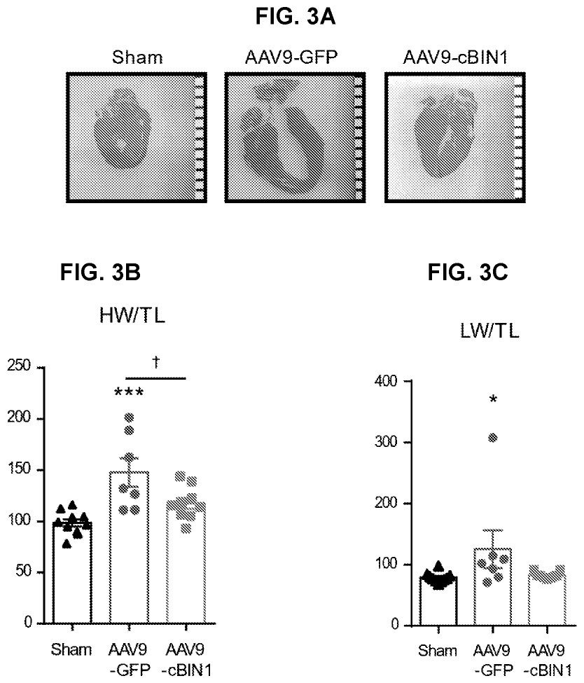

Then, the overall survival rate (non-survival is death) in all groups was explored. As indicated in the Kaplan-Meier curves in FIG. 2, the overall survival in AAV9-cBIN1 treated TAC mice (survival rate 77.8%, 14/18) lies in the middle of sham (survival rate 100%, 10/10) and AAV9-GFP treated TAC mice (survival rate 58.8%, 10/17) (p=0.0202 by log-rank test for trend when comparing across three groups) (FIG. 2A). Next, in all survived TAC mice with EF≥35% at Time 0 (5 weeks post-TAC and before virus injection), systolic HF free survival (non-survival as death or EF<35%) was further analyzed between the two viral groups during follow-up echocardiography monitoring. Systolic HF free survival is significantly improved by AAV9-cBIN1 (p=0.0225 by log-rank test when compared to the AAV9-GFP group) (FIG. 2B). Of the 11 AAV9-GFP mice with EF≥35% at Time 0, 9 developed EF<35% within 20 weeks post-TAC with 2 premature deaths and 7 with progressive EF reduction. In comparison, of the 13 AAV9-cBIN1 mice with EF≥35% at Time 0, only 5 developed EF<35% within 20 weeks post-TAC with 1 premature death. Furthermore, of these 5 mice counted as non-survival in the Kaplan-Meier curve analysis, other than the one mouse that died prematurely, the remaining 3 out of 4 non-survival AAV9-cBIN1 treated animals had EF recovery over 35% at 20 weeks post-TAC. The surviving mice were then sacrificed and evaluated for ratios of HW/TL and LW/TL. AAV9-cBIN1 mice did not have a significant increase in HW/TL and LW/TL as occurred in AAV9-GFP mice when compared to sham control mice (FIG. 3A-B). These data indicate that cBin1 gene therapy protects cardiac function and effectively increases overall survival and HF-free survival, blocking, postponing, or even reversing the worsening cycles of HF progression.

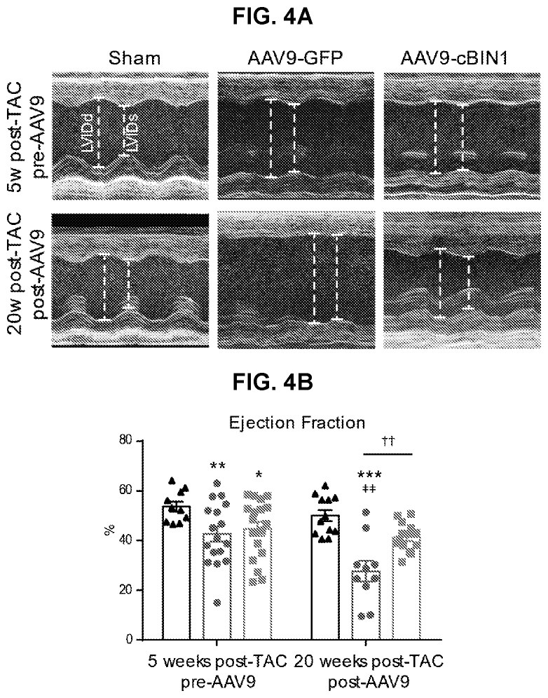

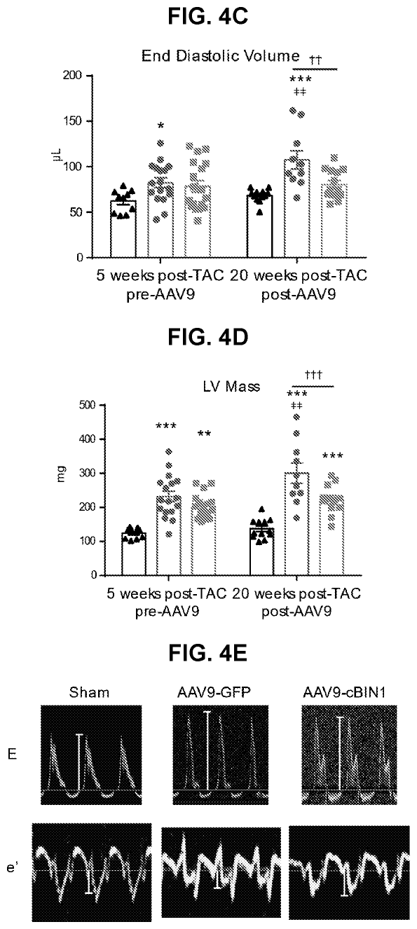

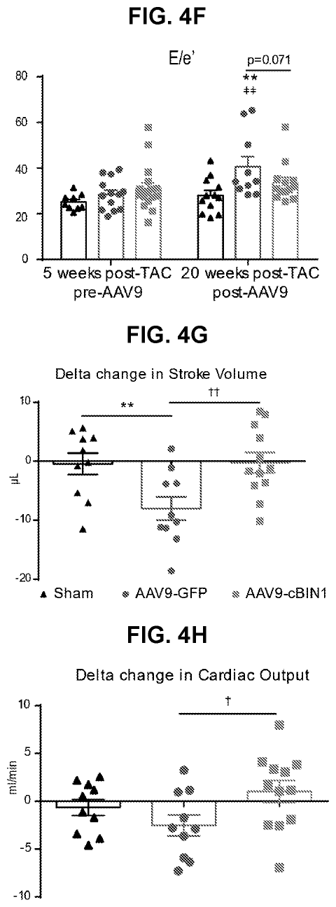

Of all the mice that survived at 20 weeks post-TAC, echocardiography measured myocardial function and physiological parameters were further compared across groups both before and after AAV9 treatment (Table 1). At 20 weeks post-TAC, AAV9-GFP mice developed significant LV contractile dysfunction (EF reduction) and chamber dilation (EDV elevation, FIG. 4A-C, Table 1), which were normalized by AAV9-cBIN1 treatment. The increase in LV mass at 20 weeks post-TAC was significantly reduced in AAV9-cBIN1 treated mice when compared to AAV9-GFP group (FIG. 4D). Furthermore, the observed delta reductions in stroke volume and cardiac output in AAV9-GFP group were also abolished in AAV9-cBIN1 treated group (FIG. 4E-F). These results indicate that cBin1 gene therapy preserves myocardial function when administered to failing hearts.

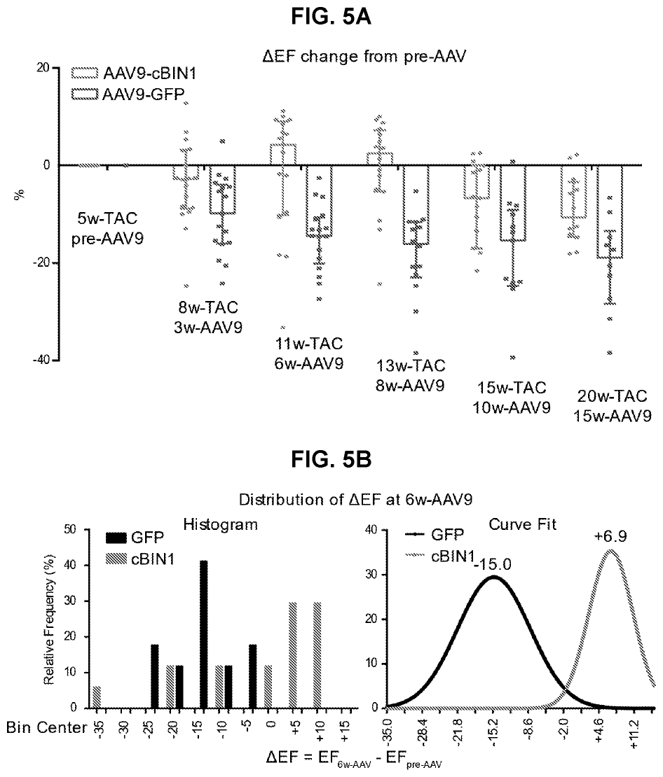

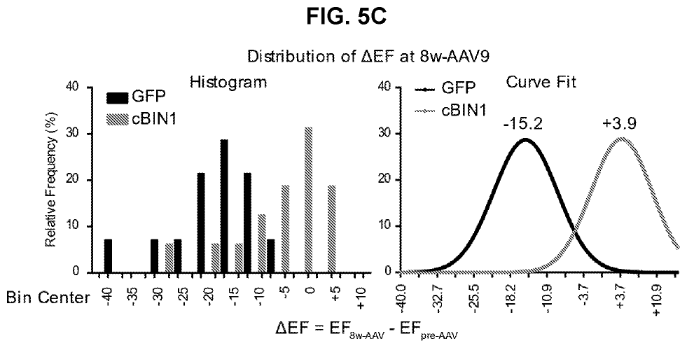

To further explore the progression of systolic dysfunction in post-TAC hearts after viral injection at Time 0 (pre-AAV9, 5 weeks post-TAC), the delta EF changes (ΔEF) from pre-AAV to 3, 6, 8, 10, 15 weeks post-AAV9 injection (correspondingly 8, 11, 13, 15, 20 weeks post-TAC) were monitored by echocardiography (FIG. 5A). Exogenous cBIN1-induced EF recovery peaked at 6 to 8 weeks post-AAV9 injection with continuous improvement of EF in the following weeks, whereas progressive EF reduction was noted in the AAV9-GFP group. The observed recovery of EF was demonstrated on the histogram distribution of AEF with Gaussian fitting (FIG. 5B-C). AAV9-cBIN1 had a right-shifted histogram distribution of AEF when compared to AAV9-GFP group. For instance, at 6 weeks post-AAV9, there is a medium EF (%) reduction of −15.0 in AAV9-GFP group, while a medium recovery of +6.9 in EF (%) was observed in the AAV9-cBIN1 group. These data indicate that exogenous cBIN1, when administered at 5 weeks post-TAC, can rescue myocardial systolic function in hearts with TAC-induced HF.

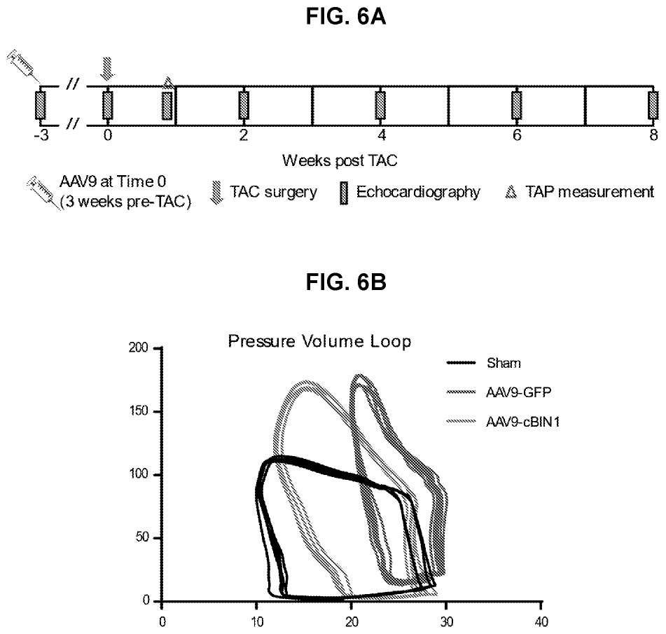

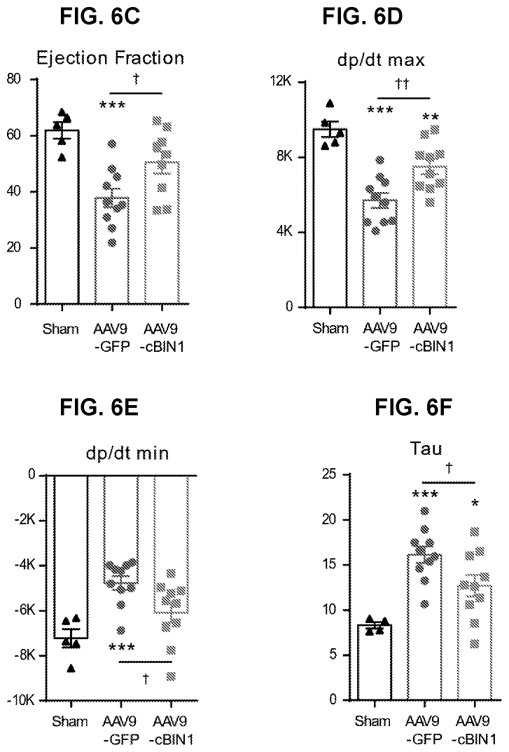

We recently reported that, in mice receiving AAV9-cBIN1 pretreatment (3×10¹⁰ vg at 3 weeks prior to TAC surgery; FIG. 6A), the incidence of TAC-induced HF is significantly reduced with a resultant better HF-free survival at 8 weeks post-TAC [34]. These data are consistent with the observed myocardial protection when AAV9 was administered after TAC surgery. To further establish the cardioprotective effect of exogenous-cBIN1 in the TAC mice, intracardiac hemodynamics were obtained in AAV9-pretreated mice using invasive PV loop recording. FIG. 6B contains representative PV loops of sham, AAV9-GFP, and AAV9-cBIN1 pretreated hearts 8 weeks after TAC surgery (FIG. 6B). The EF and the maximal rate of pressure change during systole (dp/dt max) were decreased in AAV9-GFP group, which were normalized by exogenous cBIN1 (FIG. 6C-D). When exploring the effects on relaxation kinetics, it was found that the maximal rate of pressure decay (dp/dt min) was decreased in AAV9-GFP group but was normalized by AAV9-cBIN1 (FIG. 6E). The increased time constant for isovolumic relaxation (Tau) in AAV9-GFP group was also rescued by cBin1 gene transfer (FIG. 6F), indicating improved cardiac relaxation. Together, these data indicate that exogenous cBIN1 improves cardiac inotropy and lusitropy in pressure overloaded hearts.

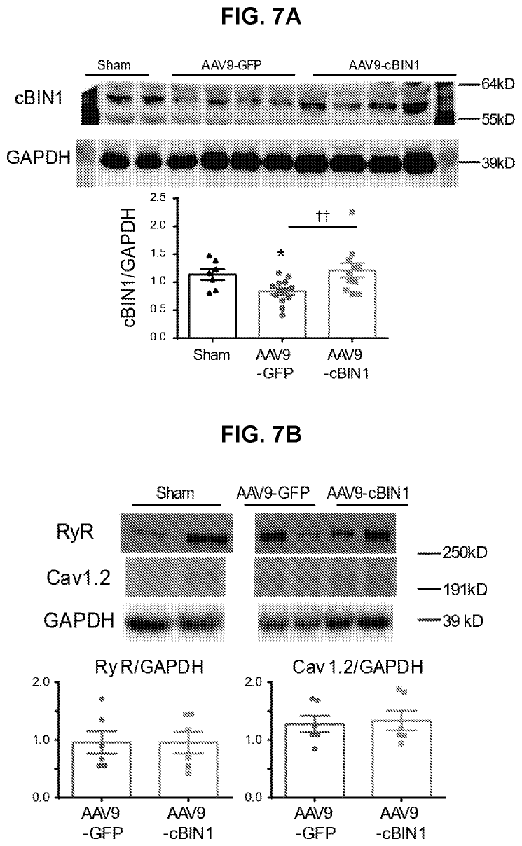

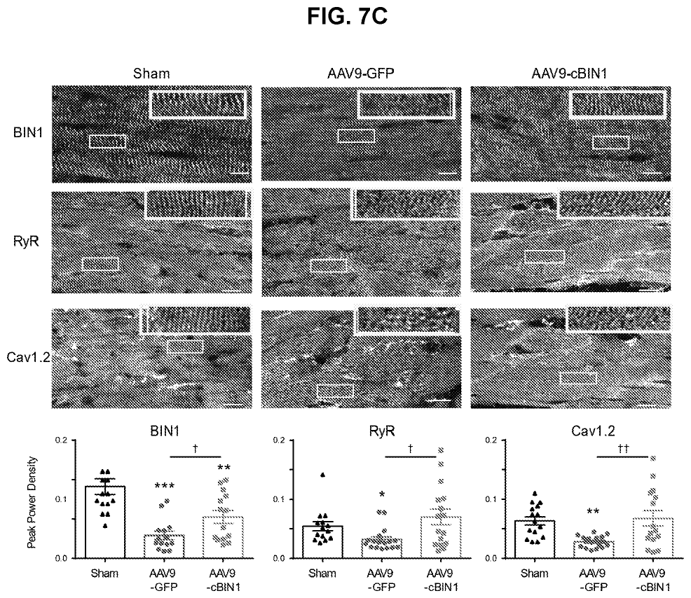

We previously identified that cBIN1 creates t-tubule microdomains and organizes LTCC-RyR dyads for efficient and dynamic regulation of cardiac function and EC coupling [25]. More recently, we found that in sympathetic overdriven mouse hearts developing diastolic dysfunction, cBIN1-microdomains are disrupted and are rescued by AAV9-cBIN1. Here, we also explored the alterations in cardiac t-tubule cBIN1-microdomains and the effect of exogenous cBIN1 in post-TAC hearts. Western blotting (FIG. 7A) identifies that myocardial cBIN1 protein is significantly reduced in mouse hearts 8 weeks after TAC (27% less than sham controls, p<0.05), which is normalized by AAV9-cBIN1 pretreatment. Along with cBIN1 rescue, membrane labeling with Di-8-ANNEPs (FIG. 17) identifies that compared to sham cardiomyocytes, 8 weeks post-TAC cardiomyocytes have a significantly reduced t-tubule cBIN1-microdomain intensity, which is normalized in cardiomyocytes from mice with AAV9-cBIN1 pretreatment. Next, immunofluorescent imaging was used to analyze the organization of cBIN1-microdomains and dyads at the myocyte level. Power spectrum analysis of BIN1 signal identifies that the well-organized t-tubule distribution of cBIN1 in sham myocardium is disrupted in post-TAC hearts, which is preserved with AAV9-cBIN1 pretreatment (FIG. 7C). Although total LTCC and RyR protein levels are not significantly altered among groups by Western blotting (FIG. 7B), myocardial distribution of LTCCs and RyRs (FIG. 7C) becomes disorganized in post-TAC hearts, which is also significantly improved in hearts with AAV9-cBIN1 pretreatment (p<0.05). These data indicate that exogenous cBIN1 normalizes TAC-induced reduction of myocardial cBIN1, resulting in preservation of cBIN1-scultped microdomains at t-tubules. Via normalizing t-tubule microdomains in pressure-overloaded hearts, cBIN1 replacement therapy thus reorganizes cardiac LTCC-RyR couplons required for beat-to-beat calcium cycling and efficient EC coupling.

### Example 3

Exogenous cBIN1 Reduces Concentric Hypertrophy in Mouse Hearts after Isoproterenol Infusion

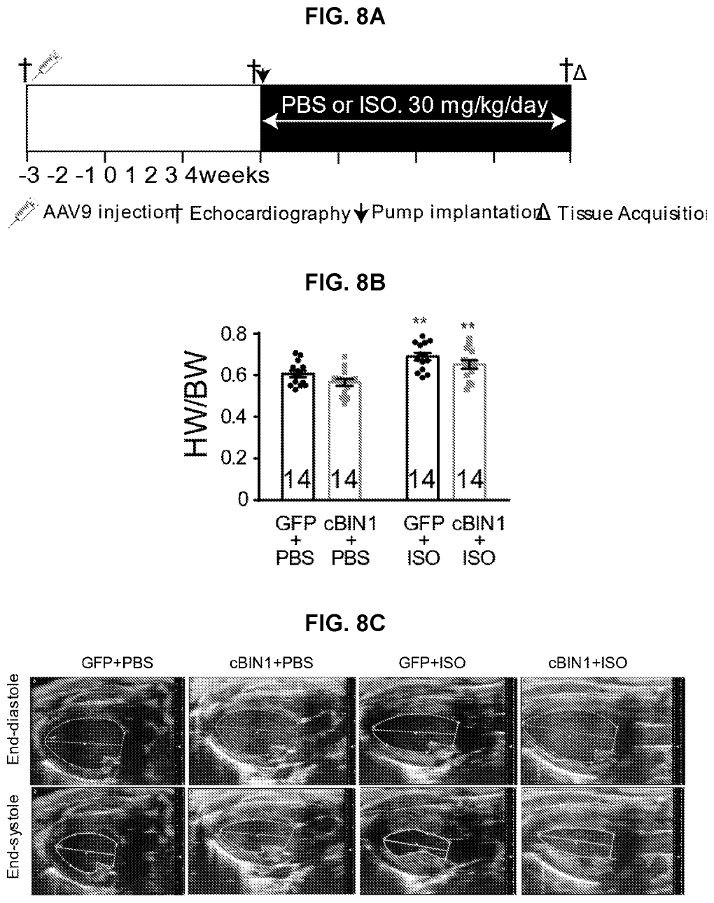

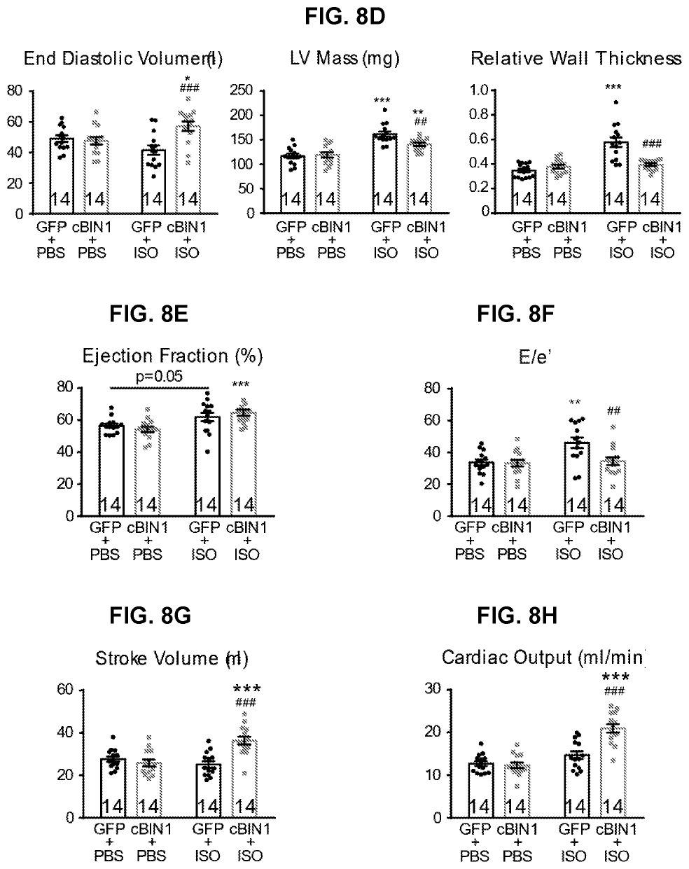

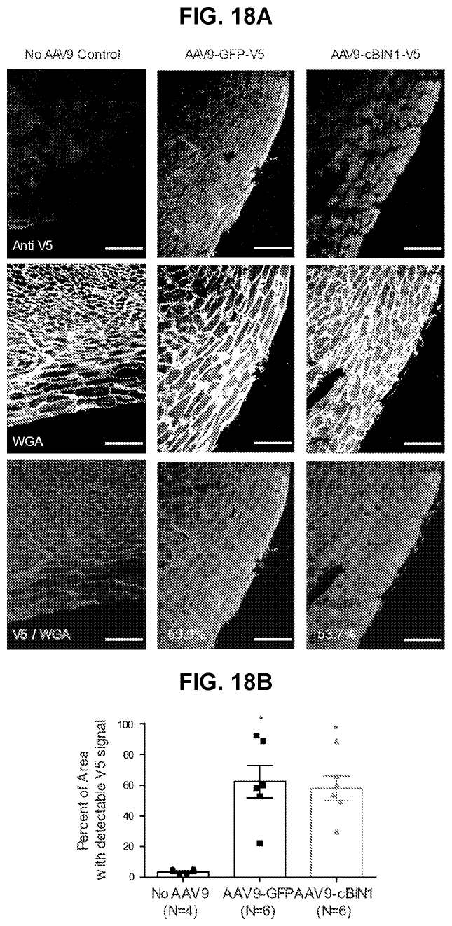

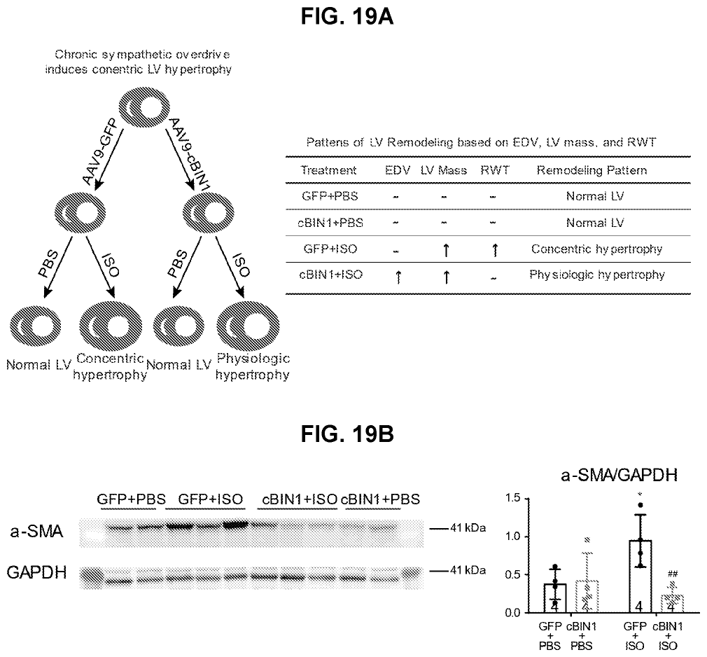

The effect of cBIN1 on myocardial function in animals subjected to 4 weeks of isoproterenol infusion was investigated (FIG. 8A). AAV9 was used to introduce myocardial expression of exogenous V5-tagged GFP or cBIN1 [74] 3 weeks prior to the onset of isoproterenol. Anti-V5 labeling identified a similar percent of myocardial area with detectable V5 signal at 7 weeks after AAV9 injection (GFP, 62.4+10.5%; cBIN1, 57.9.2+7.8%), indicating successful transduction of exogenous protein in over half of cardiomyocytes (FIG. 18). It is possible that the remaining near 40% of negatively stained cardiomyocytes may express exogenous proteins at a low level below the detection threshold of immunofluorescence. In all mice, isoproterenol significantly increased heart weight to body weight ratio (HW/BW), indicating cardiac hypertrophy (FIG. 8B). Cardiac geometry and function were assessed by echocardiography (FIG. 8C-H). In AAV9-GFP pretreated animals, isoproterenol induced a significant increase in LV mass and relative wall thickness (RWT), without altering end diastolic volume (EDV), consistent with echocardiography-based classification of concentric hypertrophy [75]. In AAV9-cBIN1 pretreated animals, the isoproterenol increase of LV mass was attenuated with a normal RWT and increased EDV, similar to echocardiography-classified “physiologic hypertrophy-like LV remodeling” using a previous reported method [75] (FIG. 19A). Furthermore, α-smooth muscle actin was increased in GFP+ISO hearts but not in cBIN1+ISO hearts (FIG. 19B), indicating AAV9-cBIN1 limits isoproterenol-induced LV hypertrophy. In AAV9-GFP pretreated mice, isoproterenol resulted in a small increase in EF (p=0.050 vs. GFP+PBS and p=0.007 vs. cBIN1+PBS group), yet onset of LV passive filling (E) to myocardial relaxation (e′) ratio (E/e′) strongly increased, indicating the onset of diastolic dysfunction. In mice pretreated with AAV9-cBIN1, isoproterenol still increased systolic function and importantly maintained a normal E/e′, indicating positive inotropy with preserved lusitropy. Furthermore, although without blood pressure measurement, isoproterenol significantly increased heart rate (HR) in all animals, indicating the effectiveness of isoproterenol in causing hemodynamic stress. Yet, post-isoproterenol HR was not different between GFP+ISO and cBIN1+ISO mice (Table 2), confirming that further improved post-isoproterenol cardiac output in AAV9-cBIN1 mice was due to muscle efficiency and not increased rate.

TABLE 2
Echocardiographic Parameters of AAV9-GFP or cBIN1 Injected Mice After 4 Weeks of PBS
or ISO Infusion

| | GFP + PBS (n = 14) | CBIN1 + PBS (n = 14) | GFP + ISO (n = 14) | cBIN1 + ISO (n = 14) |
|---|---|---|---|---|
| HW/BW (mg/g) | 6.07 + 0.15 | 5.65 + 0.17 | 6.90 + 0.18** | 6.52 + 0.20** |
| LVM (mg) | 116.87 + 4.71 | 119.19 + 5.37 | 161.18 + 5.45*** | 140.70 + 3.33**^{##} |
| RWT | 0.35 + 0.02 | 0.38 + 0.02 | 0.58 + 0.04*** | 0.39 + 0.01^{###} |
| LVEDV (μL) | 49.11 + 2.15 | 47.70 + 2.48 | 41.58 + 3.06 | 57.21 + 3.06*^{##} |
| SV (μL) | 27.64 + 1.24 | 25.81 + 1.56 | 25.13 + 1.50 | 36.37 + 1.86***^{###} |
| CO (mL/min) | 12.70 + 0.58 | 12.32 + 0.67 | 14.68 + 0.90 | 20.95 + 0.99***^{###} |
| HR (bpm) | 454.41 + 10.90 | 481.30 + 13.70 | 583.41 + 9.87*** | 569.05 + 7.10*** |
| | | | 61.98 + 2.65 | |
| EF (%) | 56.43 + 1.31 | 54.19 + 1.68 | (p = 0.05 vs. | 64.63 + 1.94*** |
| | | | GFP + PBS) | |
| Ele’ | 33.85 + 1.87 | 33.27 + 2.13 | 46.06 + 3.31** | 34.51 + 2.44^{##} |

LVM, left ventricular mass;
RWT, relative wall thickness;
LVEDV, left ventricular end-diastolic volume;
SV, stroke volume;
CO, cardiac output;
HR, heart rate;
EF, ejection fraction;
FS, fraction area shortening;
E/e’, transmitral blood flow velocity of early diastolic period/the septal mitral annulus tissue velocity of early diastolic period.
Data are expressed as Mean ± SEM.
*, **, *** indicates P < 0.05, 0.01, 0.001 respectively for comparison of PBS vs. ISO with the same AAV9 treatment;
^{#}, ^{##}, ^{###} indicates P < 0.05, 0.01, 0.001 respectively for comparison of AAV8-GFP vs. AAV9-cBIN1 with isoproterenol (ISO) infusion.

### Example 4

Chronic Isoproterenol-Disrupted cBIN1-Microdomains can be Normalized by AAV9-cBIN1

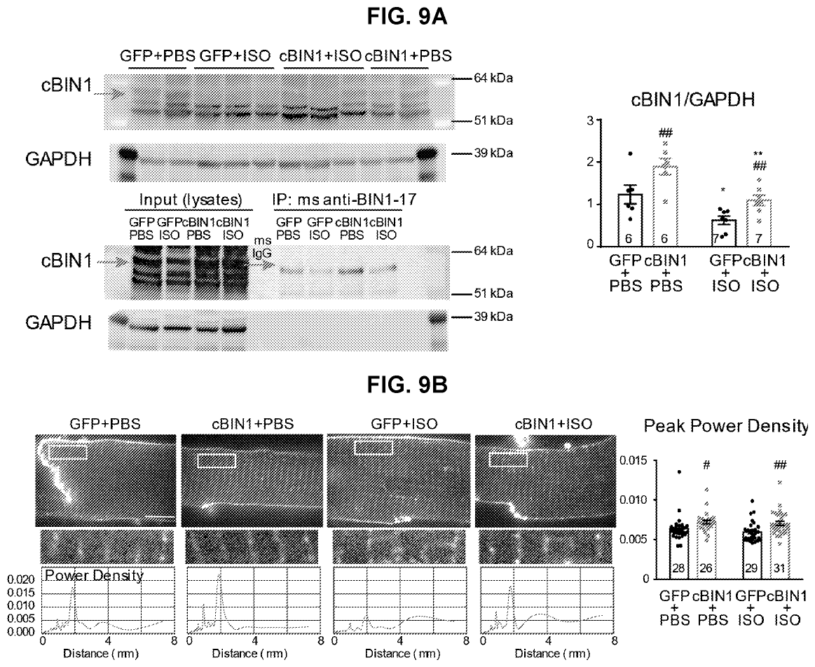

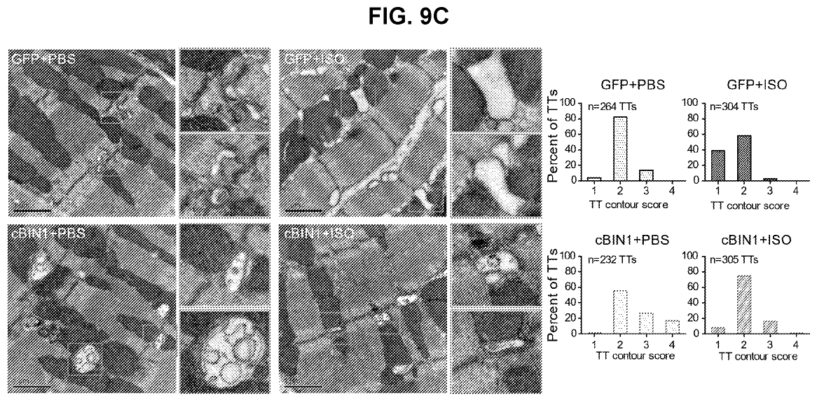

It is well known that both myocardial inotropy and lusitropy are related to cardiomyocyte calcium cycling [76]. cBIN1, the structural organizer for dyad microdomains [31], creates t-tubule microfolds to limit extracellular Ca²⁺ diffusion [29], facilitates microtubule-dependent forward trafficking of L-type calcium channels (LTCCs) [30], and clusters of LTCCs that are already delivered to t-tubule membrane. Therefore, how cBIN1-microdomains may remodel in hypertrophic hearts after chronic isoproterenol infusion was explored. Western blots of heart lysates indicate that isoproterenol induced a significant reduction in cBIN1 protein, which is normalized by AAV9-cBIN1 (FIG. 9A). Note that immunoprecipitation with anti-BIN1-exon 17 antibody followed by Western blot detection with anti-BIN1-exon 13 antibody confirms that the examined protein band is the cBIN1 (BIN1+13+17) isoform. Gross t-tubule network architecture was also examined in isolated cardiomyocytes labeled with a membrane dye Di-8-ANNEPs (FIG. 9B). Live-cell imaging followed by power spectrum analysis indicated that overall t-tubule organization (normalized peak power density) remained similar in GFP+ISO mice, and was increased by AAV9-cBIN1. Even though the gross t-tubule network remained organized, using transmission electron microscopy (TEM) imaging, it was noted that in GFP+ISO hearts there was a reduction in t-tubule microfolds, which were preserved in cBIN1+ISO hearts (FIG. 9C). Quantitation of the degree of contoured t-tubules using a modified scoring system established previously [29] (1, round or dilated t-tubule lumen without folds; 2, non-circular contoured t-tubule lumen without folds; 3, t-tubules with 2-3 layer of folds; or 4, t-tubules with>3 layer of folds) identified a significant reduction in t-tubule contour in GFP+ISO hearts, which was normalized in cBIN1+ISO hearts (p<0.001, Chi-square test). Note the exaggerated microfolds (more than 3 layers of folds, score of 4) are found in cBIN1+PBS hearts, a result of greater than physiologic levels of cBIN1. These data indicate that cBIN1 is critical for the formation of t-tubule microfolds, the folds are downregulated under chronic sympathetic overdrive, and the folds can be restored by cBIN1 exogenous therapy.

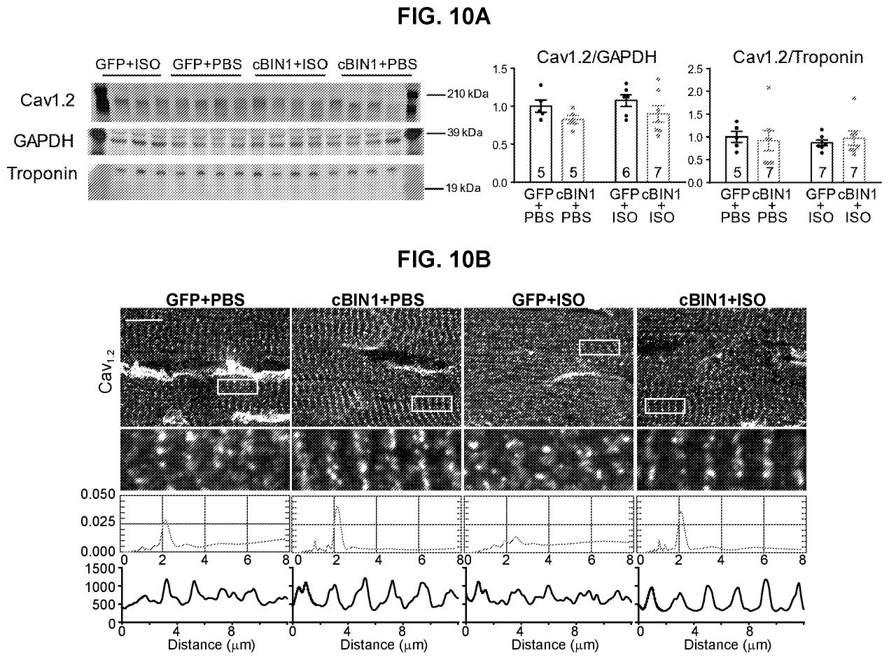

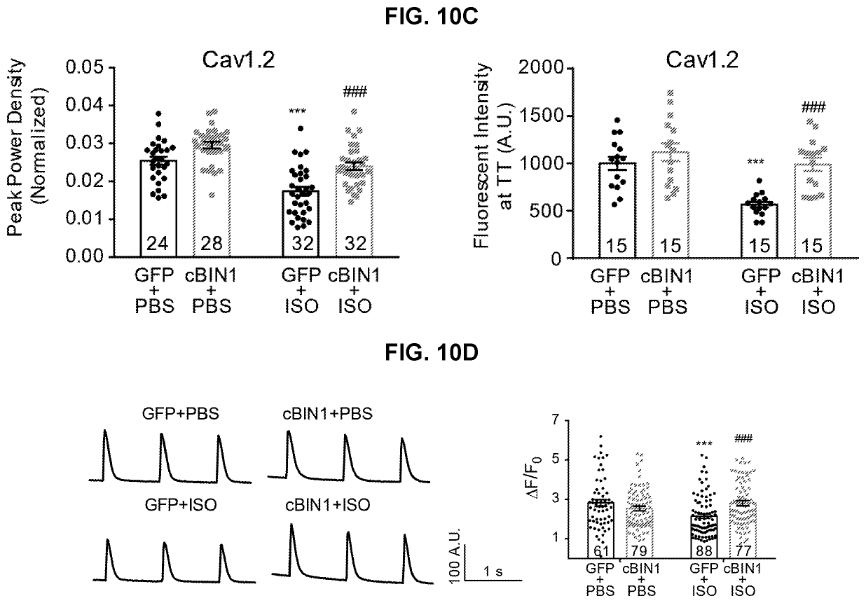

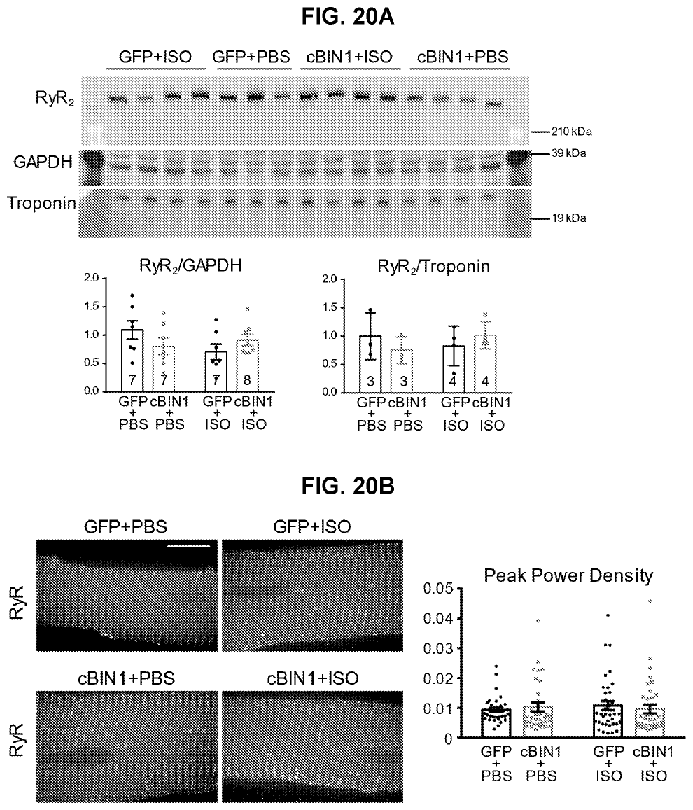

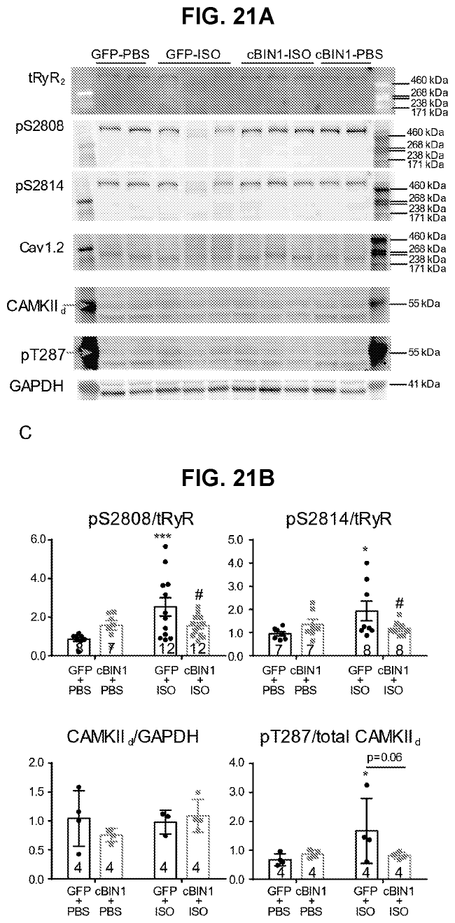

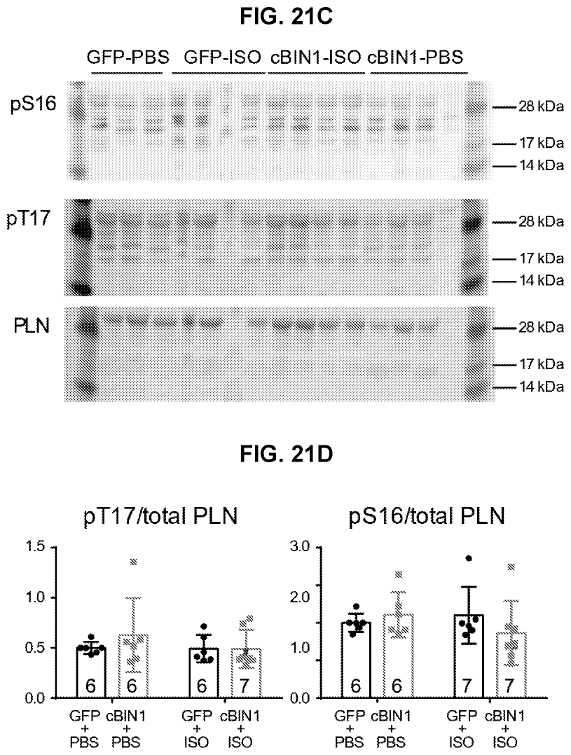

Subsequently, Cav1.2 expression and intracellular distribution in cardiomyocytes was explored. In post-isoproterenol hearts, the net myocardial protein expression of Cav1.2 was similar (FIG. 10A). However, myocardial tissue immunofluorescent labeling of Cav1.2 revealed that channel density along t-tubules was significantly reduced in GFP+ISO cardiomyocytes, which was normalized by AAV9-cBIN1 (FIG. 10B-C), power spectrum and fluorescent profile analysis). These data are consistent with previous observations of altered Cav1.2 protein distribution despite of similar total protein levels [32]. With reduced Cav1.2 localization to t-tubules, the peak amplitude of calcium transient (ΔF/F₀) was significantly reduced in GFP+ISO cardiomyocytes when compared to that from control GFP+PBS myocytes (FIG. 10D), which was normalized by exogenous cBIN1. Ryanodine receptor 2 (RyR) total protein expression and intracellular distribution were not different across all groups (FIG. 20). However, RyRs became hyper-phosphorylated at both PKA-dependent S2808 and CAMKII-dependent S2814, consistent with a previous report [61]. Together with increased phosphorylation at T287 in CAMKIIδ, these data indicate that PKA and CAMKII activation induced RyR hyperphosphorylation occur after chronic isoproterenol infusion. Importantly, AAV9-cBIN1 pretreatment successfully blunts these pathways and reduces RyR hyperphosphorylation (FIG. 21).

### Example 5

Exogenous cBIN1 Improves SERCA2a Distribution Along SR

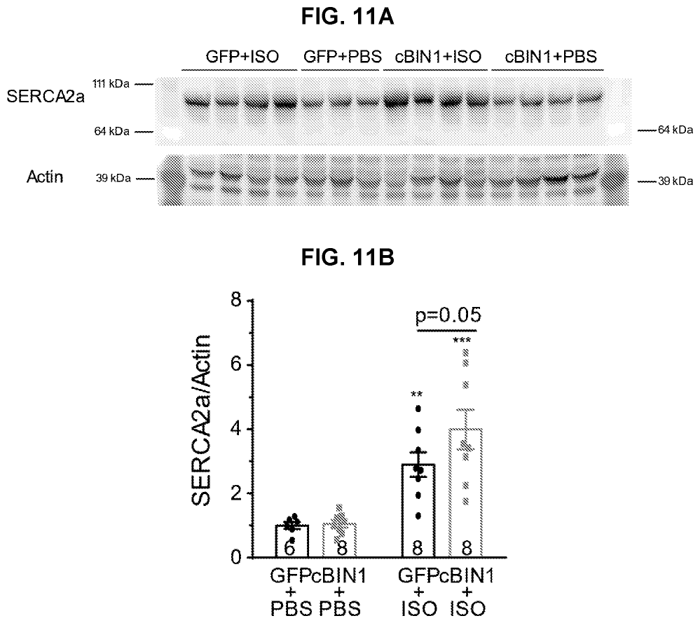

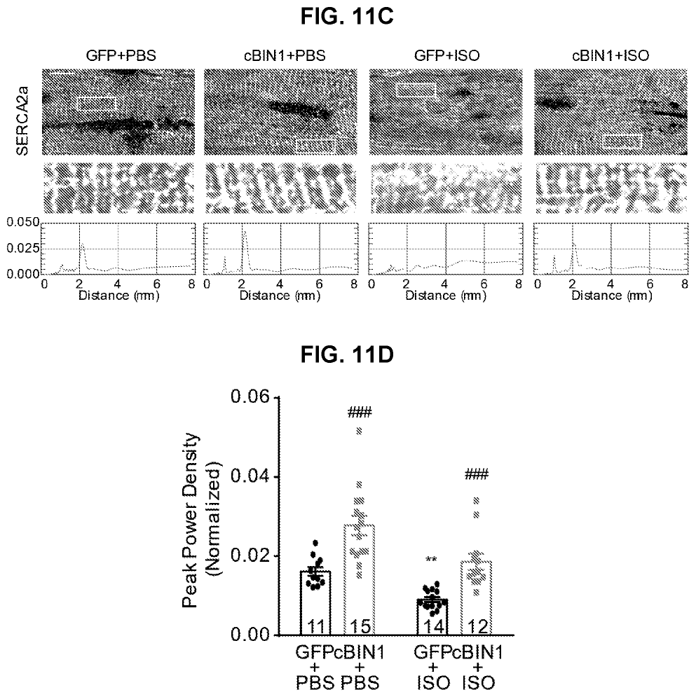

Cardiac lusitropy is most directly related to calcium reuptake via SERCA2a. Surprisingly, despite impaired diastolic dysfunction in GFP+ISO hearts, total protein expression of SERCA2a was significantly increased after isoproterenol infusion (FIG. 11A-B). Total protein levels of phospholamban (PLN) and its phosphorylated forms (pS16 and pT17) were not altered (FIG. 21). Previous studies indicated that acute isoproterenol-induced PLN phosphorylation can be normalized after chronic isoproterenol infusion and even PLN dephosphorylation can occur due to activation of serine/threonine phosphatases PP1 and PP2A [73, 77]. Consistent with these reports, the present results demonstrated that unchanged PLN phosphorylation after 4-weeks of isoproterenol infusion is a possible net result of balanced local activation of both kinases and phosphatases. These data indicate that both SERCA2a protein and activity are not decreased in post-isoproterenol hearts. Given the effect of cBIN1 on Cav1.2 localization, SERCA2a localization was examined. Myocardial tissue sections with SERCA2a labeling were imaged with spinning-disc confocal microscopy and compared across groups (FIG. 11C). In GFP+PBS hearts, a subpopulation of SERCA2a was concentrated to the t-tubule/jSR regions, giving rise to an organized distribution with a major power spectrum peak at 1.8-2 μm, corresponding to the full length of a sarcomere. Overexpression of cBIN1 in the cBIN1+PBS hearts further increased SERCA2a signals near t-tubule/jSR. In GFP+ISO hearts, intracellular distribution of SERCA2a was disorganized with a significant reduction in peak power density, which was normalized in cBIN1+ISO hearts (quantification in FIG. 11D).

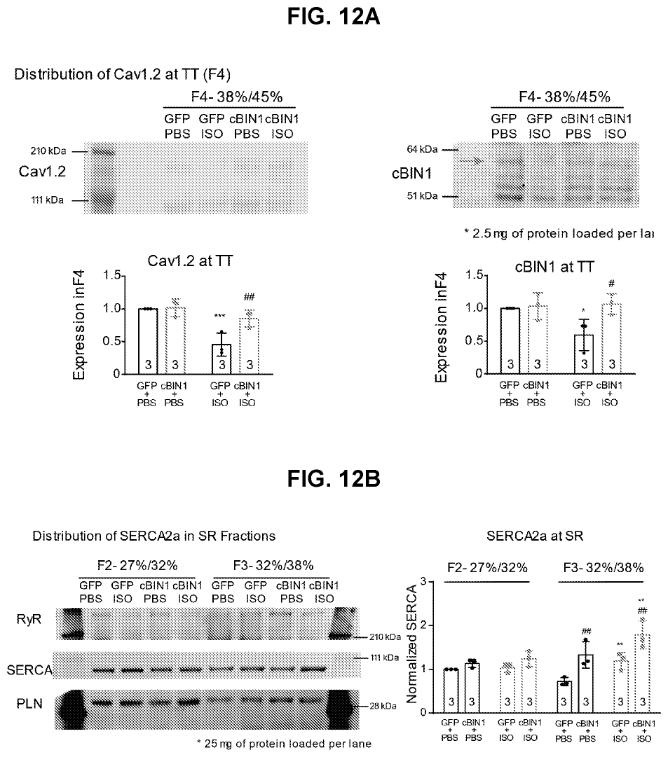

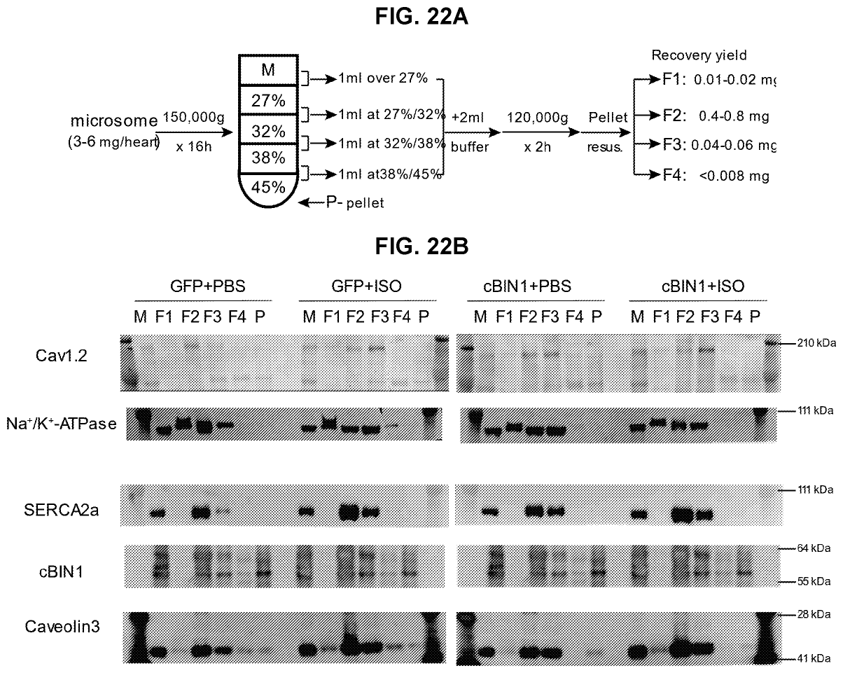

Intracellular distribution of Cav1.2 and SERCA2a was further explored using biochemical sucrose gradient-based fractionation of cardiac microsomes [70]. As indicated in FIG. 22A, fraction F4 has the lowest recovery yield when compared to other fractions. However, even with a low yield, Cav1.2 and cBIN1 are detectable in F4 with limited Na⁺/K⁺-ATPase and depleted SERCA2a, indicating F4 was enriched with t-tubule origin microsomes (FIG. 22B). When normalizing t-tubule protein concentration for F4 across all samples (2.5 μg protein loaded per lane), GFP+ISO hearts have a significant reduction in both cBIN1 and Cav1.2 protein per unit t-tubule when compared to control GFP+PBS hearts, which was normalized by AAV9-cBIN1 pretreatment (FIG. 12A). These data are consistent with immunofluorescent imaging that identified less t-tubule localization of Cav1.2 channels following isoproterenol infusion, and restoration with AAV9-cBIN1. On the other hand, SR proteins were detected only in fractions F2 and F3. When normalizing SR protein concentration for F2 and F3 (25 μg protein loaded per lane), F3 had relatively more RyR and less PLN than F2 (FIG. 12B), indicating more enrichment of jSR toward the heavier F3 fraction. Quantification of SERCA2a expression in F2 and F3 identified that when compared to AAV9-GFP, AAV9-cBIN1 caused a significant increase in SERCA2a distribution into the heavier and more jSR enriched F3, but not the longitudinal SR enriched F2 fraction (FIG. 12B). Of note, isoproterenol alone did increase SERCA2a expression in F3 in AAV9-GFP mouse hearts, likely due to an overall increase in total protein expression of SERCA2a in post-isoproterenol hearts (FIG. 11A). These data indicate that in isoproterenol infused hearts, exogenous cBIN1 can maintain t-tubule microdomains to localize Cav1.2 and SERCA2a to their functional sites.

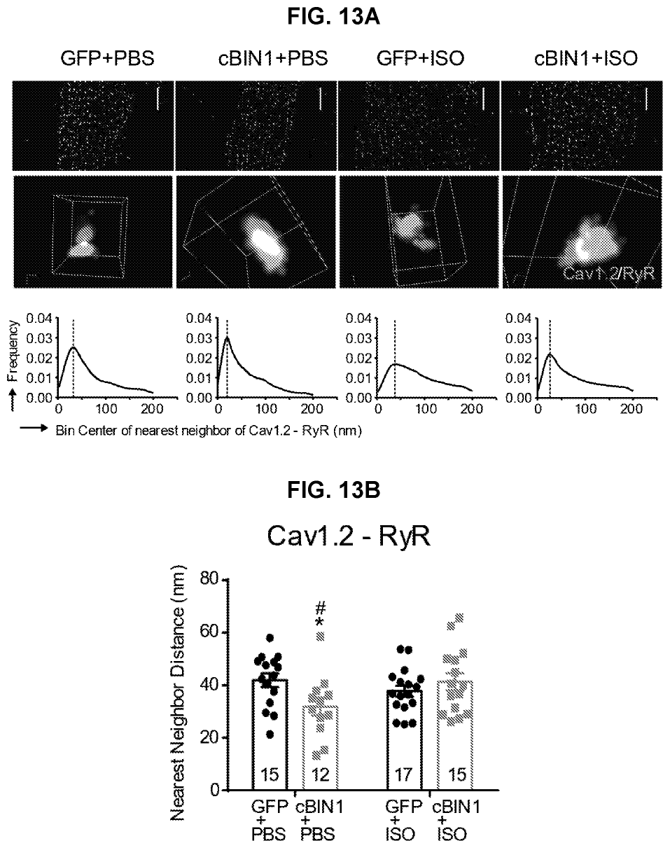

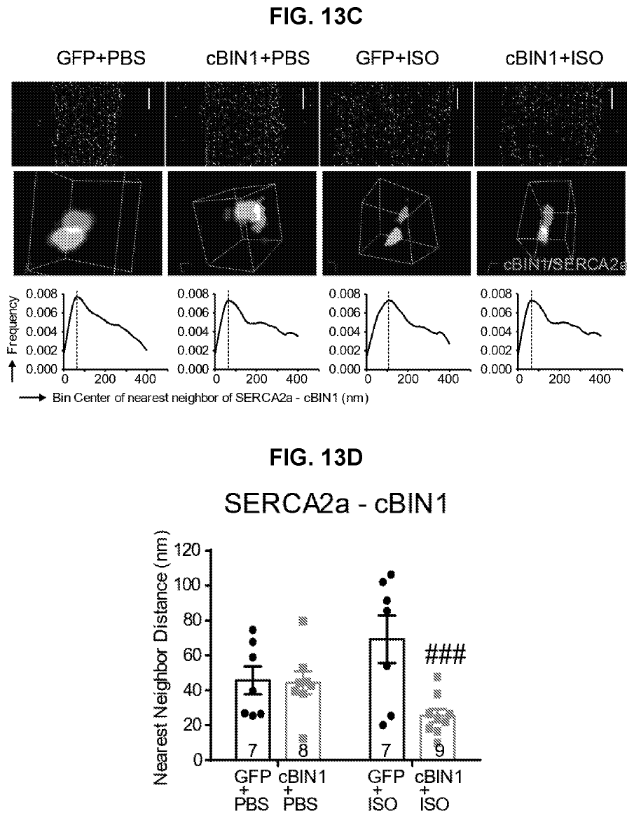

Given reduced Cav1.2 and SERCA2a at t-tubule/jSR region, STORM imaging was used to analyze nanoscale protein-protein colocalization for Cav1.2-RyR and SERCA2a-cBIN1 (FIG. 13). Using nearest neighbor analysis, the distance between individual Cav1.2 molecule and its closest RyR molecule was quantified. Histogram distribution of distances between Cav1.2-RyR molecules from whole cell images identified a first peak near 40 nm in GFP+PBS, GFP+ISO, and cBIN1+ISO cardiomyocytes, corresponding to dyad couplons. In GFP+ISO hearts with preserved systolic function, the distribution histogram tended to shift to the right yet with still preserved first peak position (FIG. 13B). Interestingly, cBIN1+PBS myocytes had a left shifted histogram distribution and a significantly reduced Cav1.2-RyR peak distance, indicating tightened couplons likely brought closer by exaggerated cBIN1-microfolds as observed in TEM imaging. On the other hand, the distance between SERCA2a and its nearest neighbor cBIN1 at t-tubules had a trend of increase after isoproterenol in AAV9-GFP pretreated animals (p=0.063, GFP+PBS vs. GFP+ISO), which was significantly decreased in AAV9-cBIN1 pretreated animals (p<0.001, GFP+ISO vs. cBIN1+ISO) (FIG. 13C-D). These data indicate that cBIN1-microfolds can regulate colocalization and interaction between EC coupling and calcium handling proteins.

### Example 6

The Phenotype of cBIN1+ISO Hearts is Isoform Specific and Unique to cBIN1

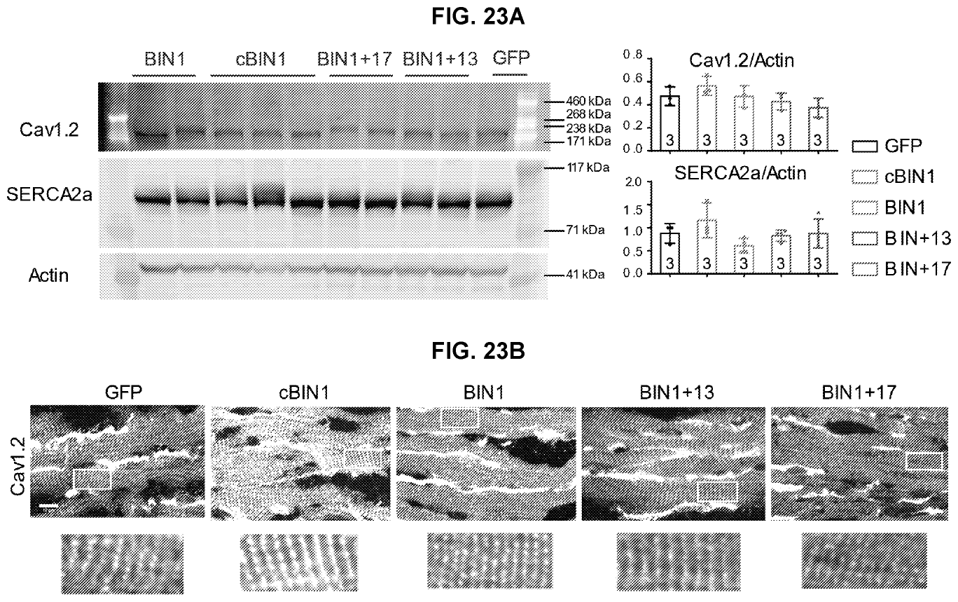

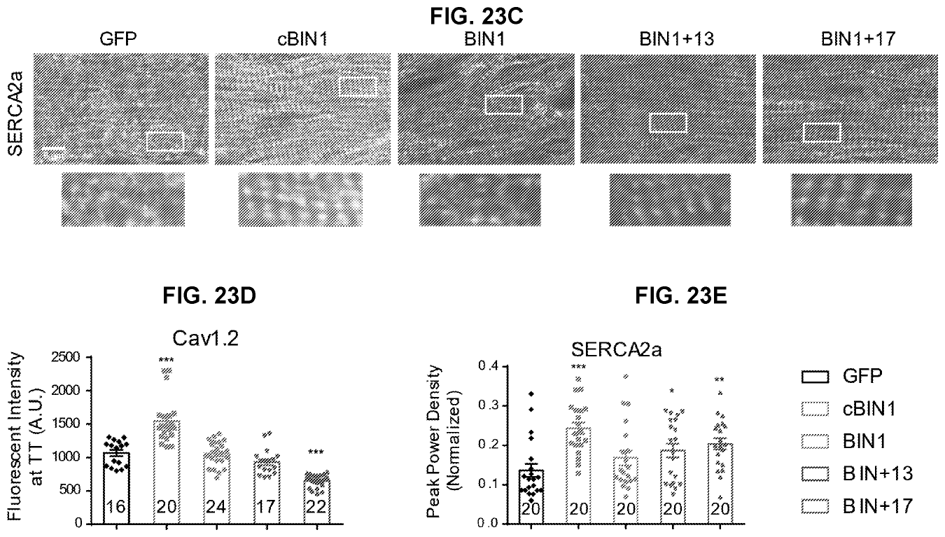

To further explore whether the observed phenotype of cBIN1+ISO hearts was an isoform specific effect, the isoproterenol protocol was repeated in 50 more mice, which were randomized to receive AAV9 transducing GFP and cBIN1, as well as the other three mouse cardiomyocyte expressing BIN1 isoforms including the small BIN1, BIN1+17, and BIN1+13. Similarly, three weeks after viral administration, mice were subjected to continuous subcutaneous isoproterenol infusion at 30 mg/kg/day for 4 weeks. The protein expression of Cav1.2 and SERCA2a in post- isoproterenol hearts were not significantly different when compared across five groups of mice transduced with GFP or BIN1 isoforms (FIG. 23A). Myocardial tissue immunofluorescent labeling of Cav1.2 channels revealed that channel density along t-tubules was significantly increased only in cBIN1-expressing hearts, but not the other BIN1 isoforms (FIG. 23B, quantification in FIG. 23D). Immunofluorescent imaging revealed that exogenous cBIN1 introduced by AAV9 organized SERCA2a distribution (FIG. 23C, quantification in FIG. 23E), consistent with the data from FIG. 11.

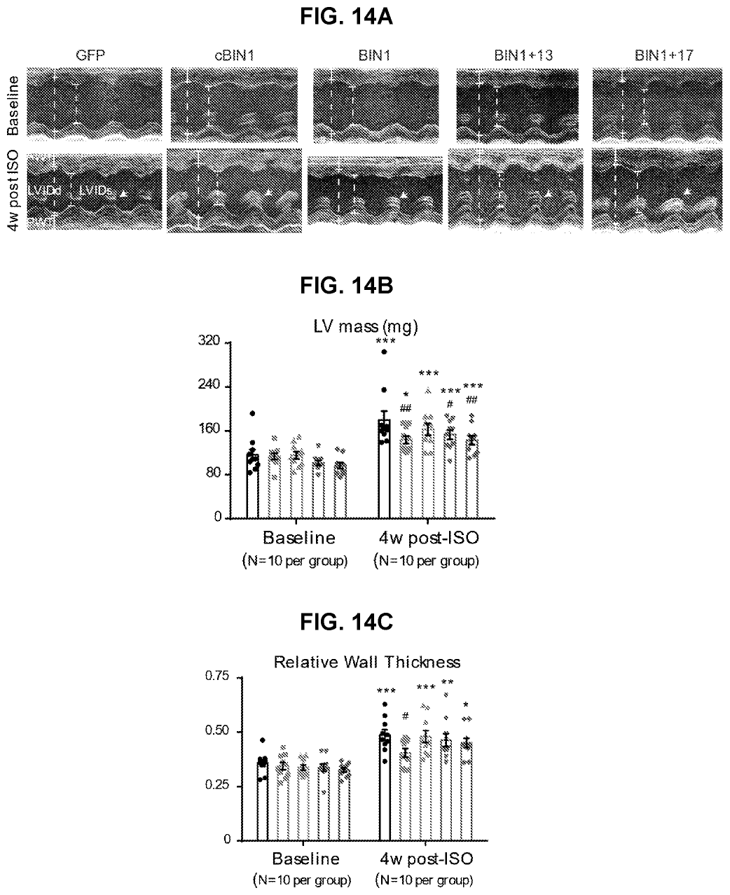

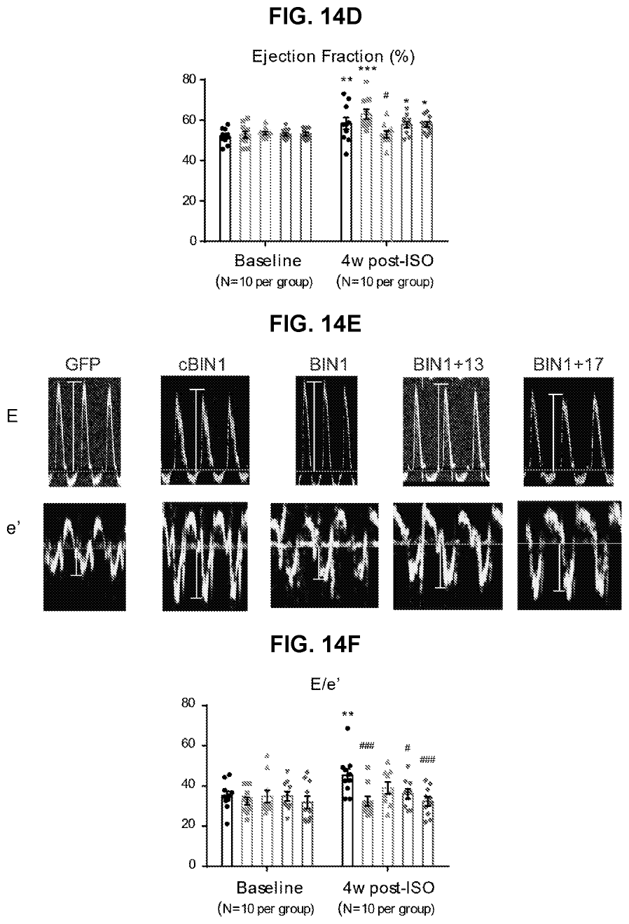

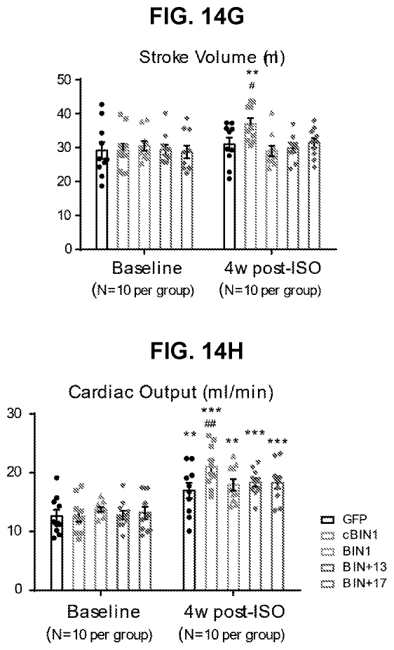

Next, the functional consequence of different AAV9-BIN1 isoform pretreatment was explored using echocardiography. cBIN1-expressing mice, when compared to the GFP group, lessened the isoproterenol induced increase in LV wall thickness, LV mass, and RWT (FIG. 14A-D, Table 3). In all animals, post-isoproterenol cardiac output is significantly increased from its level at baseline, a result from isoproterenol-induced increase in heart rate RWT (Table 3). Yet only cBIN1 hearts also have an improved systolic function, normalized E/e′, increased stroke volume, and further increased cardiac output when compared to post-isoproterenol GFP hearts (FIG. 14E-H). Of note, a partial cBIN1-like effect occurred in mice pretreated with BIN1+17, which significantly reduced LV mass, E/e′, and attenuated isoproterenol-increased RWT. The observed partial diastolic functional rescue from BIN1+17 was consistent with the partial rescue of intracellular distribution of SERCA2a by immunofluorescent imaging (FIG. 23C). However, due to the inability of BIN1+17 to increase Cav1.2 distribution at t-tubules, there was not a positive inotropic effect in AAV9-BIN1+17 pretreated hearts following isoproterenol infusion.

TABLE 3
Echocardiographic Parameters of AAV9-GFP or BIN1 isoforms-
Injected Mice Before and After ISO Infusion

| | Baseline | | | | | | | |
|---|---|---|---|---|---|---|---|---|
| | | | | BIN1 + | BIN1 | Pre-ISO | | |
| | GFP | cBIN1 | BIN1 | 13 | + 17 | GFP | cBIN1 | BIN1 |
| LVM (mg) | 116.49 ± | 113.33 ± | 115.52 ± | 101.51 ± | 96.78 ± | 106.55 ± | 114.35 ± | 113.69 ± |
| | 9.80 | 5.70 | 6.57 | 4.38 | 5.38 | 5.28 | 4.48 | 6.36 |
| RWT | 0.36 ± | 0.34 ± | 0.34 ± | 0.34 ± | 0.33 ± | 0.33 ± | 0.36 ± | 0.32 ± |
| | 0.02 | 0.02 | 0.01 | 0.02 | 0.01 | 0.02 | 0.02 | 0.01 |
| LVEDV (μL) | 56.67 ± | 55.96 ± | 57.54 ± | 55.45 ± | 54.63 ± | 57.38 ± | 55.92 ± | 54.45 ± |
| | 4.39 | 4.38 | 1.94 | 2.02 | 3.32 | 2.98 | 2.73 | 2.43 |
| SV (μL) | 29.15 ± | 29.16 ± | 30.49 ± | 29.33 ± | 28.69 ± | 29.04 ± | 29.03 ± | 28.92 ± |
| | 2.47 | 1.95 | 1.38 | 1.43 | 1.85 | 1.75 | 1.70 | 1.39 |
| CO (mL/min) | 12.67 ± | 12.63 ± | 13.77 ± | 12.77 ± | 13.18 ± | 13.04 ± | 14.19 ± | 14.06 ± |
| | 1.03 | 0.95 | 0.38 | 0.75 | 1.05 | 0.98 | 0.90 | 0.92 |
| HR (bpm) | 443 ± | 443 ± | 484 ± | 471 ± | 449 ± | 446 ± | 488 ± | 484 ± |
| | 19 | 12 | 7 | 12 | 12 | 15 | 12 | 15 |
| EF (%) | 52.02 ± | 52.75 ± | 53.49 ± | 52.96 ± | 53.05 ± | 50.66 ± | 51.84 ± | 53.19 ± |
| | 1.22 | 1.66 | 0.78 | 0.82 | 0.82 | 1.46 | 1.62 | 1.00 |
| FS (%) | 17.01 ± | 16.49 ± | 18.70 ± | 17.53 ± | 18.18 ± | 17.53 ± | 18.48 ± | 19.48 ± |
| | 0.80 | 1.60 | 1.17 | 1.13 | 1.05 | 1.28 | 1.58 | 0.89 |
| E/e′ | 35.14 ± | 32.53 ± | 34.83 ± | 34.88 ± | 31.88 ± | 37.23 ± | 34.07 ± | 32.73 ± |
| | 2.21 | 1.89 | 3.07 | 2.32 | 3.11 | 3.12 | 2.75 | 3.00 |

| | Pre-ISO | | 4 w post-ISO | | | | |
|---|---|---|---|---|---|---|---|
| | BIN1 + | BIN1 + | | | | BIN1 + | BIN1 + |
| | 13 | 17 | GFP | cBIN1 | BIN1 | 13 | 17 |
| LVM (mg) | 113.96 ± | 106.66 ± | 179.82 ± | 143.67 ± | 162.89 ± | 152.89 ± | 142.87 ± |
| | 4.05 | 3.59 | 16.14*** | 6.92*^{##} | 10.92*** | 8.43*** | 8.20***^{##} |
| RWT | 0.33 ± | 0.32 ± | 0.49 ± | 0.41 ± | 0.48 ± | 0.46 ± | 0.45 ± |
| | 0.02 | 0.01 | 0.02*** | 0.02*^{##} | 0.03*** | 0.03*** | 0.02*** |
| LVEDV (μL) | 54.21 ± | 51.18 ± | 54.76 ± | 61.34± | 55.19 ± | 51.81 ± | 53.04 ± |
| | 2.63 | 2.34 | 4.95 | 4.34 | 3.47 | 3.28 | 2.51 |
| SV (μL) | 29.53 ± | 27.50 ± | 31.00 ± | 36.94 ± | 28.98 ± | 29.78 ± | 31.35 ± |
| | 1.57 | 1.26 | 1.88 | 1.66**^{#} | 1.53 | 1.19 | 1.36 |
| CO (mL/min) | 14.07 ± | 12.53 ± | 16.95 ± | 21.02 ± | 17.92 ± | 18.33 ± | 18.32 ± |
| | 1.08 | 0.72 | 1.31** | 1.13***^{#} | 0.99** | 0.73*** | 1.03*** |
| HR (bpm) | 472 ± | 455 ± | 546 ± | 568 ± | 572 ± | 589 ± | 582 ± |
| | 17 | 14 | 25*** | 13*** | 23*** | 12*** | 11*** |
| EF (%) | 54.43 ± | 53.79 ± | 58.40 ± | 62.95 ± | 52.97 ± | 57.71 ± | 57.93 ± |
| | 1.09 | 1.13 | 2.97** | 2.44*** | 1.64^{#} | 1.53 | 1.33 |
| FS (%) | 17.39 ± | 17.17 ± | 18.30 ± | 20.95 ± | 18.13 ± | 18.90 ± | 18.37 ± |
| | 1.90 | 1.60 | 1.46 | 1.58* | 1.79 | 1.25 | 1.08 |
| E/e′ | 34.12 ± | 36.96 ± | 45.33 ± | 32.39 ± | 39.06 ± | 36.07 ± | 32.33 ± |
| | 3.88 | 2.19 | 3.20* | 2.37^{##} | 2.97 | 2.47 | 2.28^{##} |

LV geometry and function were evaluated by echocardiography at baseline, before isoproterenol infusion (Pre-ISO) and 4 weeks post isoproterenol treatment (4 w post-ISO). LVM, left ventricular mass; RWT, relative wall thickness; LVEDV, left ventricular end-diastolic volume; SV, stroke volume; CO, cardiac output; HR, heart rate; EF, ejection fraction; FS, fraction area shortening; E/e′, transmitral blood flow velocity of early diastolic period/the septal mitral annulus tissue velocity of early diastolic period.
Data are expressed as Mean ± SEM.
*,
**,
***indicates P < 0.05, 0.01, 0.001 vs. the baseline of each own group;
^{#},
^{##},
^{###}indicates P < 0.05, 0.01, 0.001 vs. the GFP group of 4 w post-ISO.

### Example 7

The Cardiac Protective Effect of AAV9-cBIN1 is Confirmed in TAC-Induced HF

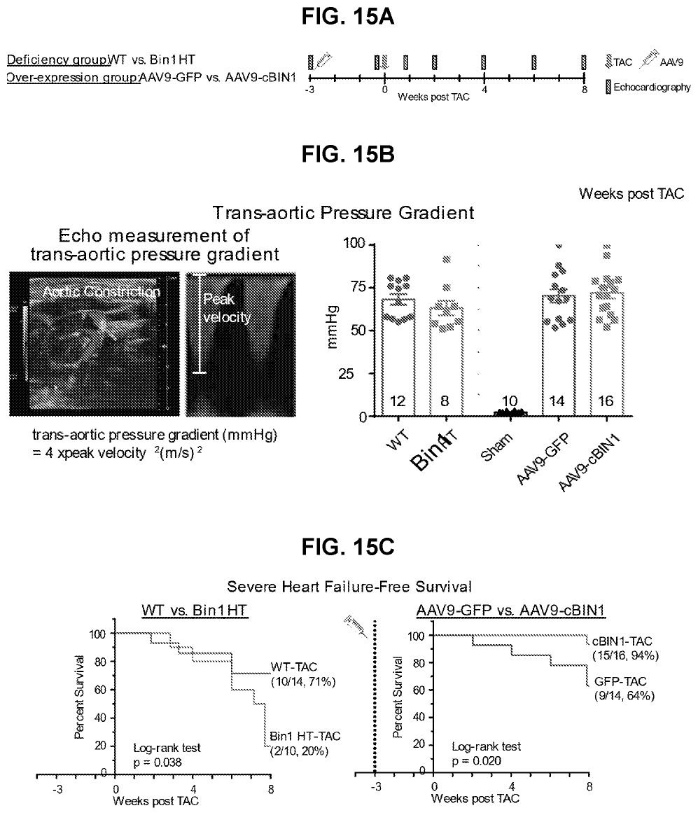

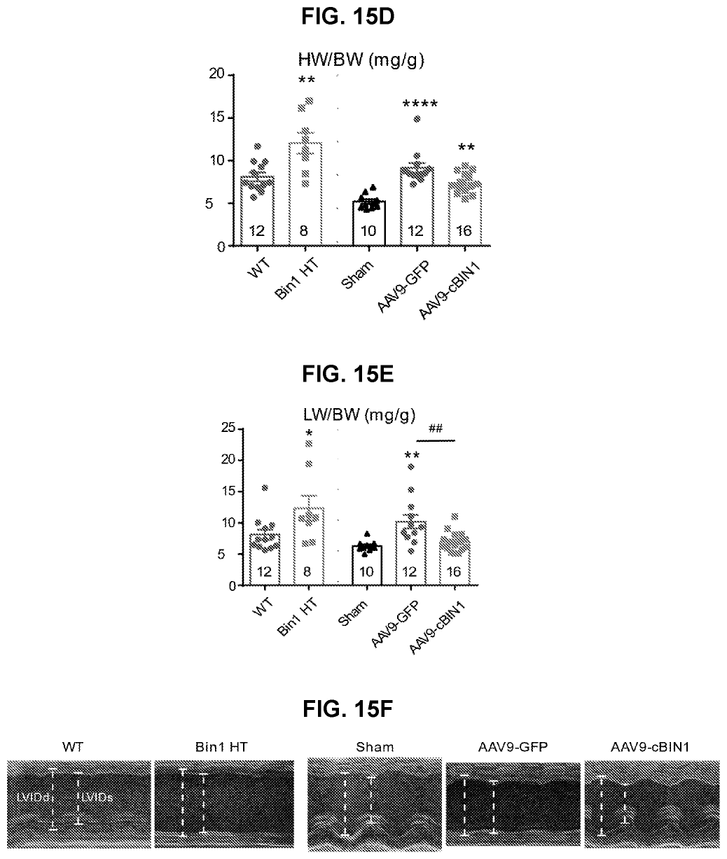

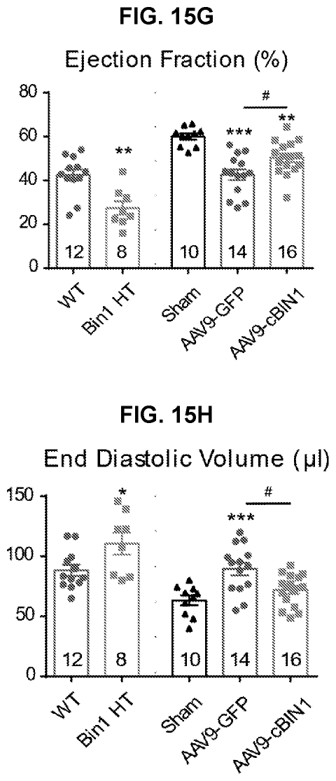

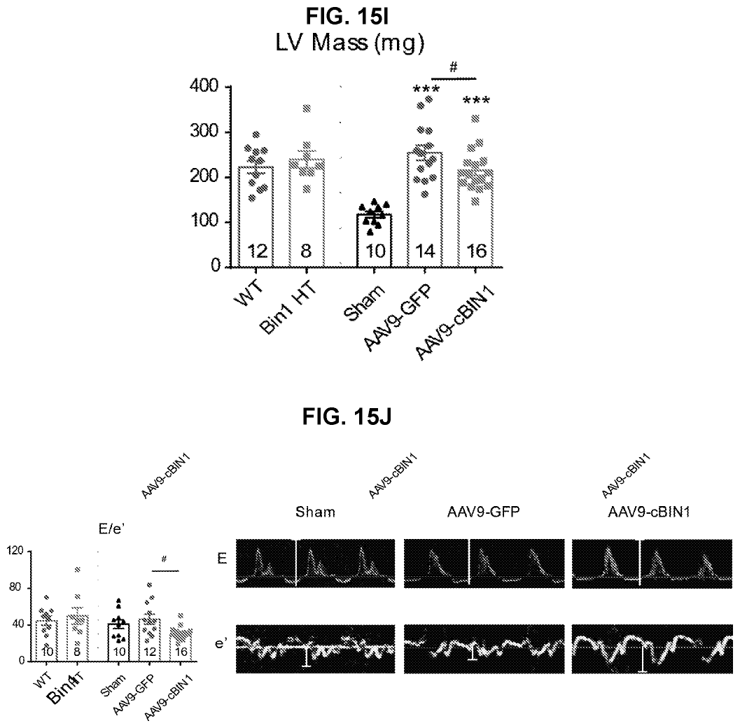

The myocardial protective effect of cBIN1 was further explored in a separate mouse model of pressure overload induced by TAC. Mice with either genetic deficiency of cBIN1 or AAV9- transuced cBIN1 over-expression were tested in this study (FIG. 15A). The deficiency study involved cardiac specific Bin1 HT mice and WT littermate controls [29], both subjected to TAC for 8 weeks. The over-expression study involved mice subjected to 8 weeks of TAC with prior injection of AAV9 transducing cBIN1-V5 or AAV9-GFP-V5, and mice subjected to an open-chest mock surgery (sham). Mice were monitored and terminated at 8 weeks post-surgery. The viruses (AAV9-GFP/cBIN1-V5), dosage (3×10¹⁰ vg), administration time (3 weeks prior to surgery), and route (retro-orbital injection) were the same as those used in the isoproterenol study. Aortic constriction in all TAC mice was confirmed by elevated trans-aortic pressure gradient (FIG. 15B), establishing a similar increase in hemodynamic afterload in all mice receiving TAC. Kaplan-Meier curves summarizing severe HF-free (EF≥35%) survival rates were included in FIG. 15C. The rate of survival in Bin1 HT mice was 20.0% (8 of 10, 2 deaths and 6 EF<35%), which was decreased from 71.4% (non-survival 4 of 14, 1 death and 3 EF<35%) in WT mice (p=0.038 by log-rank test). As expected, all sham mice survived through the entire experimental protocol (10 of 10, dotted black line). The rate of survival was decreased to 64.3% in the AAV9-GFP mice (non-survival 5 of 14, 5 EF<35%), which was significantly improved to 93.7% in the AAV9-cBIN1 group (non-survival 1 of 16, 1 EF<35%) (p=0.020 by log-rank test). These data indicate that higher cBIN1 protein content in the heart is associated with better survival without development of systolic HF following pressure overload.

At 8 weeks after TAC, surviving mice were sacrificed and evaluated for ratios of HW/BW and LW/BW (Table 4, FIG. 15D-E). Both HW/BW and LW/BW were significantly higher in Bin1 HT mice verses WT mice, indicating worsening of LV hypertrophy and lung edema with BIN1 deficiency. Regarding gene therapy, AAV9-cBIN1 significantly reduced LW/BW from that of the control GFP-TAC group to the level of sham hearts, representing a striking reduction in TAC-induced pulmonary edema. Hypertrophy still occurred in the AAV9-cBIN1 hearts, although to a lesser extent. These data indicated that exogenous cBIN1 reduced TAC-induced hypertrophy and prevented deterioration into HF. Echocardiography analysis (FIG. 15F-J, Table 5) further indicated that when compared to WT-TAC mice, there was a significant decrease in EF and increase in EDV in Bin1 HT-TAC hearts, indicating worsening of dilated cardiomyopathy with BIN1 deficiency. On the other hand, AAV9-cBIN1 significantly reduced TAC-induced LV dilation (EDV) and contractile dysfunction (EF), limiting the development of dilated cardiomyopathy. As a result, AAV9-cBIN1 pretreatment-maintained stroke volume and cardiac output in post-TAO hearts without hearts being dilated. Furthermore, tissue doppler identified that the diastolic parameter E/e′ values of both lateral and septal wall were significantly improved in AAV9-cBIN1 pretreated hearts, indicating better diastolic function in mice with exogenous cBIN1. These data indicate that cBin1 gene therapy preserves myocardial systolic and diastolic function in stressed hearts, and effectively prevents the development of dilated cardiomyopathy in mouse hearts subjected to pressure overload.

TABLE 4
Phenotype Characteristics in All Mice 8 Weeks after Sham or TAC Surgery

| | | | | AAV9-GFP | AAV9-CBIN1 |
|---|---|---|---|---|---|
| Parameter | WT (n = 12) | Bin1 HT (n = 8) | Sham (n = 10) | (n = 14) | (n = 16) |
| Pressure Gradient | 68.16 ± 3.07 | 62.77 ± 7.54 | 2.22 ± 0.22 | 70.21 ± 3.96* | 71.94 ± 3.35* |
| (mmHg) | | | | | |
| BW(g) | 29.66 ± .47 | 26.41 ± .86* | 29.66 ± 0.48 | 29.89 ± 0.48 | 30.48 ± 0.50 |
| HW(g) | 0.26 ± 0.02 | 0.32 ± 0.03 | 0.16 ± 0.01 | 0.27 ± 0.01* | 0.23 ± 0.01* |
| LW(g) | 0.24 ± 0.02 | 0.32 ± 0.04* | 0.19 ± 0.01 | 0.29 ± 0.03* | 0.21 ± 0.01 |
| HW/BW (mg/g) | 8.10 ± 0.51 | 12.07 ±1.22** | 5.20 ± 0.28 | 9.16 ± 0.58* | 7.41 ± 0.32* |
| LW/BW (mg/g) | 8.11 ± 0.80 | 2.32 ± 2.03* | 6.27 ± 0.28 | 10.19 ± 1.10* | 6.94 ± 0.40^{#} |

Data are expressed as Mean ± SEM.
*indicates p < 0.05 vs. WT or Sham;
^{#}indicates p < 0.05 for AAV9-GFP vs. AAV9-CBIN1.
BW: body weight;
HW: heart weight;
LW: lung weight.

TABLE 5
Echocardiographic Parameters in All Mice 8 Weeks after Sham or TAC Surgery

| | | | | AAV9-GFP | AAV9-CBIN1 |
|---|---|---|---|---|---|
| Parameter | WT (n = 12) | Bin1 HT (n = 8) | Sham (n = 10) | (n = 14) | (n = 16) |
| HR (bpm) | 515.70 ± 14.65 | 516.9 ± 14.56 | 511.84 ± 18.22 | 550.31 ± 9.73 | 552.32 ± 14.19 |
| EF (%) | 42.54 ± 2.59 | 27.50 ± 3.15** | 60.04 ± 1.38 | 42.69 ± 2.43* | 50.36 ± 1.91*^{#} |
| FS (%) | 21.20 ± 1.48 | 12.96 ± 1.65* | 29.78 ± 0.78 | 21.06 ± 1.40* | 25.44 ± 1.16^{#} |
| EDV (μL) | 88.25 ± 4.68 | 110.60 ± 9.23* | 63.35 ± 4.06 | 89.54 ± 5.30* | 71.97 ± 3.35^{#} |
| ESV (μL) | 51.96 ± 4.49 | 81.15 ± 9.74* | 24.59 ± 2.62 | 51.05 ± 3.46* | 35.89 ± 2.26^{#} |
| SV (μL) | 36.30 ± 1.99 | 29.47 ± 2.31* | 38.76 ± 1.88 | 38.49 ± 3.48 | 36.08 ± 2.01 |
| CO (ml/min) | 18.24 ± 1.11 | 15.53 ± 1.16 | 19.08 ± 0.76 | 20.77 ± 2.01 | 19.29 ± 0.92 |
| LVAWd (mm) | 1.04 ± 0.06 | 0.97 ± 0.06 | 0.83 ± 0.05 | 1.16 ± 0.05* | 1.12 ± 0.06* |
| LVAWs (mm) | 1.36 ± 0.07 | 1.24 ± 0.07 | 1.16 ± 0.04 | 1.42 ± 0.05* | 1.44 ± 0.08* |
| LVIDd (mm) | 4.50 ± 0.09 | 4.87 ± 0.16* | 3.92 ± 0.10 | 4.50 ± 0.13* | 4.13 ± 0.10 |
| LVIDs (mm) | 3.60 ± 0.12 | 4.36 ± 0.17* | 2.74 ± 0.16 | 3.69 ± 0.11* | 3.14 ± 0.09^{#} |
| LVPWd (mm) | 1.16 ± 0.07 | 1.10 ± 0.11 | 0.78 ± 0.04 | 1.14 ± 0.03* | 1.16 ± 0.06* |
| LVPWs (mm) | 1.48 ± 0.08 | 1.24 ± 0.11* | 1.19 ± 0.07 | 1.33 ± 0.06 | 1.46 ± 0.05* |
| LV Mass (mg) | 222.63 ± 13.75 | 239.87 ± 18.98 | 117.39 ± 7.00 | 254.48 ± 16.81* | 215.70 ± 11.40*^{#} |
| E/e’ lateral | 44.36 ± 7.89 | 49.73 ± 6.64 | 34.26 ± 4.15 | 44.30 ± 4.62 | 28.81 ± 1.96^{#} |
| E/e’ septal | 44.70 ± 4.89 | 49.93 ± 8.89 | 40.94 ± 4.77 | 46.31 ± 5.32 | 30.64 ± 1.78 |

Data are expressed as Mean ± SEM.
*indicates p < 0.05 vs. WT or Sham;
^{#}indicates p < 0.05 for AAV9-GFP vs. AAV9-cBIN1.
HR: heart rate;
EF: ejection fraction;
FS: fractional shortening;
EDV: end diastolic volume;
ESV, end systolic volume;
SV: stroke volume;
CO, cardiac output;
LVAWd/LVAWs: left ventricular anterior wall in diastole/systole;
LVIDd/LVIDs: left ventricular internal diameter in diastole/systole;
LVPWd/LVPWs: left ventricular posterior wall in diastole/systole;
E/e’ lateral: transmitral blood flow velocity of early diastolic period/the lateral mitral annulus tissue velocity of early diastolic period;
E/e’ septal: transmitral blood flow velocity of early diastolic period/the septal mitral annulus tissue velocity of early diastolic period.

### Example 8

Mouse Diabetic Cardiomyopathy (HFpEF) Studies

Myocardial function was explored in db/db mice developing diabetic cardiomyopathy, as well as the therapeutic potential of AAV9-cBIN1. The Db/db mouse line (homozygous Dock7m for Leprdb, Jackson Laboratories), an established mouse model of type 2 diabetes, has been used as a model of diabetic cardiomyopathy and heart failure with preserved ejection fraction (HFpEF) [78]. Nine-weeks old male and female db/db mice and their littermate control db/m mice were administered with one dose (1×10¹¹ vg) of AAV9-cBIN1 or control GFP through retro-orbital injection [79, 80]. Cardiac function and physiological parameters were measured both prior to AAV injection and 8 weeks after injection when animals are 17 weeks of age [79]. Myocardial function was evaluated by echocardiography-measured systolic parameters (ejection fraction and fractional shortening), diastolic parameters (E/A, E/e′), as well as stroke volume (SV). We also evaluated the performance on exercise exhaustion test of these animals by measuring their maximal running distance on a mouse treadmill. Exercise intolerance is an important physiological parameter of impaired cardiac functional reserve and HFpEF.

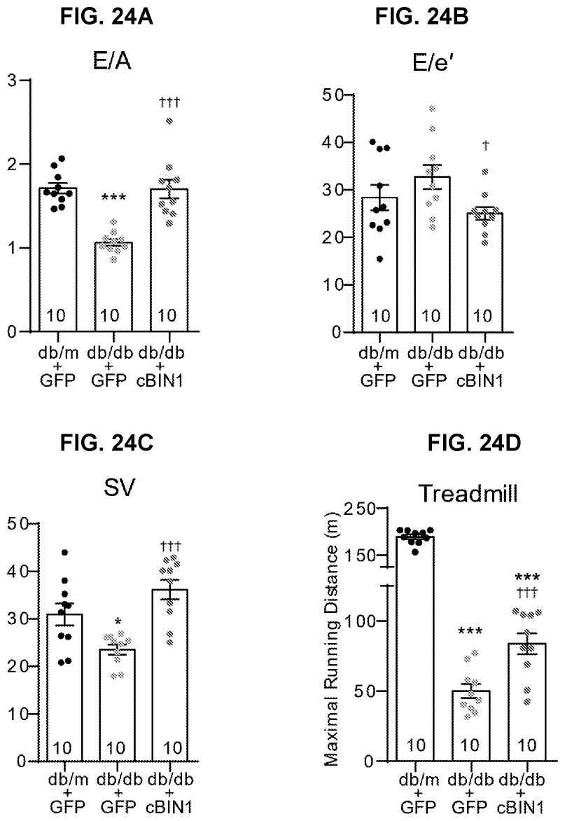

Echocardiography measured myocardial functional parameters indicate a successful development of diastolic failure as early as in 9 weeks of age in db/db mice. In 17-week old db/db mice with still preserved left ventricular ejection fraction, there is a significant alteration in diastolic parameters including reduced E/A (FIG. 23A) and elevated E/e′ (FIG. 24B). All these abnormal diastolic parameters can be rescued and normalized with treatment of AAV9-cBIN1. With AAV9-cBIN1 improved diastolic function, reduced left ventricular stroke volume in db/db mice is normalized (FIG. 24C). Along with improved echocardiography parameters, exercise intolerance in these diseased db/db mice (reduced maximal running distance on treadmill) is also significantly improved with AAV9-cBIN1 (FIG. 24D). Note the body weight of all db/db mice is similar across both groups with either AAV9-GFP or cBIN1 treatment, indicating similar levels of obesity and type 2 diabetes. Taken together with the rescue of echocardiography measured diastolic parameters, these data indicate that AAV9-cBIN1 mediated rescue effect on exercise tolerance is due to improved myocardial functional reserve.

### Example 9

Summary of Canine Ischemic Cardiomyopathy (HFrEF) Studies

We explored the effect of cBIN1 gene therapy to rescue diminished cardiac function in hearts with ischemic cardiomyopathy. Adult canine hound dogs (25-30 kg) were subjected to open thoracotomy and permanent ligation of the proximal Left Anterior Descending (LAD) coronary artery. Dogs were followed by echocardiography, hemodynamic parameters, and physiologic parameters 8 to 9 weeks post ligation, the animals were anesthetized, and their left ventricular endocardium was injected with cBIN1 packaged in AAV9 virus. The NOGA XP (Biosense Webster/Johnson and Johnson) was used to perform 3D electroanatomical mapping of the LV endocardial chamber. Using the same NOGA XP system, we inject the myocardium with a Myostar catheter that has a 27-gauge nitinol needle. Each heart was injected at 20 injections sites throughout left ventricular endocardium. Each injection consisted of 2.5×10¹¹ vg mixed in 250 pL of PBS, for a total of 5×10¹² vg per animal heart. Animals were then continued to be monitored by echocardiography, hemodynamic, and physiological parameters.

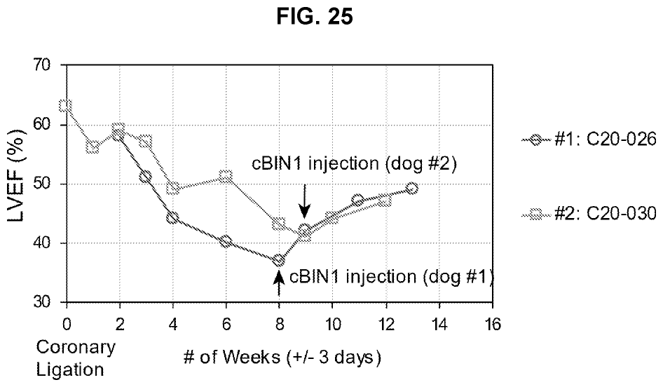

As shown in FIG. 25, we report measured left ventricular ejection fraction (LVEF) versus week of study. Data for two animals were included. Time 0 corresponds to time of LAD ligation. Arrows indicated the time of cBIN1 therapy. Note LVEF decreased by a half prior to cBIN1 therapy, and then recovers to only mild dysfunction within 1-2 weeks post injection. The first dog continues to recover. At 12 weeks, the second dog was terminated due to ingesting a foreign body which resulted in ischemic colon.

In both animals, cBIN1 gene therapy provided a dramatic rescue of myocardial function in heart with ischemic cardiomyopathy (HFrEF). Rescue occurred for at least five weeks. The experiments are ongoing, and duration of therapy after a single episode of cBIN1 injection remains to be determined.

### REFERENCES

1. Roger, “Epidemiology of heart failure,” Circulation Res. 113: 646-659 (2013).

2. Virani et al. “Heart Disease and Stroke Statistics-2020 Update: A Report from the American Heart Association,” Circulation 141: e139-e596 (2020).

3. Gomez et al., “Defective excitation-contraction coupling in experimental cardiac hypertrophy and heart failure,” Science 276: 800-806 (1997).

4. Perreault et al., Abnormalities in intracellular calcium regulation and contractile function in myocardium from dogs with pacing-induced heart failure,” J. Clin. Invest. 89: 932-938 (1992).

5. Landstrom et al., “Calcium Signaling and Cardiac Arrhythmias,” Circ. Res. 120: 1969-1993 (2017).

6. Lopez-Crisosto et al., “Sarcoplasmic reticulum-mitochondria communication in cardiovascular pathophysiology,” Nat. Rev. Cardiol. 14: 342-360 (2017).

7. Bers, “Calcium cycling and signaling in cardiac myocytes,” Annu. Rev. Physiol. 70, 23-49 (2008).

8. Lyon et al., “Loss of T-tubules and other changes to surface topography in ventricular myocytes from failing human and rat heart,” Proc. Natl. Acad. Sci. USA 106, 6854-6859 (2009).

9. Louch et al., “There goes the neighborhood: pathological alterations in T-tubule morphology and consequences for cardiomyocyte Ca2+ handling,” J. Biomed. Biotechnol. 503906 (2010).

10. Wei et al. T-tubule remodeling during transition from hypertrophy to heart failure. Circ. Res. 107: 520-531 (2010).

11. Litwin et al., “Dyssynchronous Ca(²⁺) sparks in myocytes from infarcted hearts,” Circ. Res. 87: 1040-1047 (2000).

12. Erkasap, “SERCA in genesis of arrhythmias: what we already know and what is new?” Anadolu Kardiyol. Derg. 7 Suppl 1: 43-46 (2007).

13. Houser et al., “Functional properties of failing human ventricular myocytes,” Trends Cardiovasc. Med. 10: 101-107 (2000).

14. Marx et al., “PKA phosphorylation dissociates FKBP12.6 from the calcium release channel (ryanodine receptor): defective regulation in failing hearts,” Cell 101: 365-376 (2000).

15. Periasamy and Huke, “SERCA pump level is a critical determinant of Ca(2+)homeostasis and cardiac contractility,” J. Mol. Cell. Cardiol. 33: 1053-1063 (2001).

16. Santulli et al., “Mitochondrial calcium overload is a key determinant in heart failure,” Proc. Natl. Acad. Sci. USA 112: 11389-11394 (2015).

17. Kinnally et al., “Is mPTP the gatekeeper for necrosis, apoptosis, or both?” Biochim. Biophys. Acta 1813: 616-622 (2011).

18. Nakayama et al., “Ca2+− and mitochondrial-dependent cardiomyocyte necrosis as a primary mediator of heart failure,” J. Clin. Invest. 117: 2431-2444 (2007).

19. Odagiri et al., “Local control of mitochondrial membrane potential, permeability transition pore and reactive oxygen species by calcium and calmodulin in rat ventricular myocytes,” J. Mol. Cell. Cardiol. 46: 989-997 (2009).

20. Zhou et al., “Mitochondrial dysfunction in pathophysiology of heart failure,” J. Clin. Invest. 128: 3716-3726 (2018).

21. Nikolaev et al., “Beta2-adrenergic receptor redistribution in heart failure changes cAMP compartmentation,” Science 327:1653-1657 (2010).

22. Ibrahim et al., “The structure and function of cardiac t-tubules in health and disease,” Proc. Biol. Sci. 278: 2714-2723 (2011).

23. Schobesberger et al., “T-tubule remodelling disturbs localized beta2-adrenergic signalling in rat ventricular myocytes during the progression of heart failure,” Cardiovasc. Res. 113: 770-782 (2017).

24. Balycheva et al., “Microdomain-specific localization of functional ion channels in cardiomyocytes: an emerging concept of local regulation and remodelling,” Biophys. Rev. 7: 43-62 (2015).

25. Hong and Shaw, “Cardiac T-Tubule Microanatomy and Function,” Physiol. Rev. 97:227-252 (2017).

26. Glukhov et al., “Direct Evidence for Microdomain-Specific Localization and Remodeling of Functional L-Type Calcium Channels in Rat and Human Atrial Myocytes,” Circulation 132: 2372-2384 (2015).

27. Sanchez-Alonso et al., “Microdomain-Specific Modulation of L-Type Calcium Channels Leads to Triggered Ventricular Arrhythmia in Heart Failure,” Circ. Res. 119: 944-955 (2016).

28. Bers, “Cardiac excitation-contraction coupling,” Nature 415: 198-205 (2002).

29. Hong et al., “Cardiac BIN1 folds T-tubule membrane, controlling ion flux and limiting arrhythmia,” Nat. Med. 20: 624-632 (2014).

30. Hong et al., “BIN1 localizes the L-type calcium channel to cardiac T-tubules,” PLoS Biol. 8: e1000312 (2010).

31. Fu et al., “Isoproterenol Promotes Rapid Ryanodine Receptor Movement to Bridging Integrator 1 (BIN1)-Organized Dyads,” Circulation 133: 388-397 (2016).

32. Hong et al., “BIN1 is reduced and Cav1.2 trafficking is impaired in human failing cardiomyocytes,” Heart Rhythm 9: 812-820 (2012).

33. Caldwell et al., “Dependence of cardiac transverse tubules on the BAR domain protein amphiphysin II (BIN-1),” Circ. Res.115: 986-996 (2014).

34. Liu, et al., “In Mice Subjected to Chronic Stress, Exogenous cBIN1 Preserves Calcium Handling Machinery and Cardiac Function,” JACC: Basic Translat. Sci. 5: 561-578 (2020).

35. Fu and Hong, “BIN1 regulates dynamic t-tubule membrane,” Biochim. Biophys. Acta 1863:1839-1847 (2016).

36. Hong et al., “BIN1 is reduced and Cav1.2 trafficking is impaired in human failing cardiomyocytes,” Heart Rhythm 9: 812-820 (2012).

37. Zhou and Hong, “Cardiac BIN1 (cBIN1) is a regulator of cardiac contractile function and an emerging biomarker of heart muscle health,” Sci. China Life Sci. 60: 257-263 (2017).

38. Xu et al., “The ESCRT-III pathway facilitates cardiomyocyte release of cBIN1-containing microparticles,” PLoS Biol. 15: e2002354 (2017).

39. Nikolova et al., “Association of the plasma cardiac bridging integrator 1 score, a novel diagnostic biomarker, with heart failure with preserved ejection fraction and cardiovascular hospitalizations,” JAMA Cardiology 3: 1206-1210 (2018).

40. Blaufarb and Sonnenblick, “The renin-angiotensin system in left ventricular remodeling,” Am. J. Cardiol. 77: 8C-16C (1996).

41. Liu et al., “Peroxisome proliferator-activated receptor beta/delta activation in adult hearts facilitates mitochondrial function and cardiac performance under pressure-overload condition,” Hypertension 57, 223-230 (2011).

42. Zhang et al., “Critical Roles of STAT3 in beta-Adrenergic Functions in the Heart,” Circulation 133: 48-61 (2016).

43. Zhou et al., “The Relationship between Galectin-3 and Different Patterns of Ventricular Geometry Remodelling in Aortic Valve Stenosis,” Heart Lung Circ. 25: 371-377 (2016).

44. Rincon et al., “Gene therapy for cardiovascular disease: advances in vector development, targeting, and delivery for clinical translation,” Cardiovasc. Res. 108: 4-20 (2015).

45. Bass-Stringer et al., “Adeno-Associated Virus Gene Therapy: Translational Progress and Future Prospects in the Treatment of Heart Failure,” Heart Lung Circ. 27:1285-1300 (2018)

46. Tilemann et al., “Gene therapy for heart failure,” Circ. Res. 110: 777-793 (2012).

47. Thai et al., “Cardiac-specific Conditional Knockout of the 18-kDa Mitochondrial Translocator Protein Protects from Pressure Overload Induced Heart Failure,” Sci. Rep. 8: 16213 (2018).

48. Osadchii, “Cardiac hypertrophy induced by sustained beta-adrenoreceptor activation: pathophysiological aspects,” Heart Fail Rev. 12: 66-86 (2007).

49. Soltysinska et al., “Myocardial structural, contractile and electrophysiological changes in the guinea-pig heart failure model induced by chronic sympathetic activation,” Exp. Physiol. 96: 647-63 (2011).

50. Balakumar et al., “Rodent models of heart failure,” J. Pharmacol. Toxicol. Meth. 56: 1-10 (2007).

51. Frey et al., “Hypertrophy of the heart: a new therapeutic target?” Circulation 109: 1580-1589 (2004).

52. Oliveira et al., “Cardiac anti-remodelling effect of aerobic training is associated with a reduction in the calcineurin/NFAT signalling pathway in heart failure mice,” J. Physiol. 587: 3899-3910 (2009).

53. Neves et al., “Exercise training in hypertension: Role of microRNAs,” World J. Cardiol. 6: 713-27 (2014).

54. Ooi et al., “The therapeutic potential of miRNAs regulated in settings of physiological cardiac hypertrophy,” Future Med. Chem. 6: 205-222 (2014).

55. MacDonnell et al., “Improved myocardial beta-adrenergic responsiveness and signaling with exercise training in hypertension,” Circulation 111: 3420-3428 (2005).

56. Hoydal et al., “Exercise training reverses myocardial dysfunction induced by CaMKIIdeltaC overexpression by restoring Ca²⁺ homeostasis,” J. Appl. Physiol. (1985) 121: 212-220 (2016).

57. Pitsavos et al., “The impact of moderate aerobic physical training on left ventricular mass, exercise capacity and blood pressure response during treadmill testing in borderline and mildly hypertensive males,” Hellenic. J. Cardiol. 52: 6-14 (2011).

58. Dieberg et al., “Clinical outcomes and cardiovascular responses to exercise training in heart failure patients with preserved ejection fraction: a systematic review and meta-analysis,” J. Appl. Physiol. (1985) 119: 726-733 (2015).

59. Yang et al., “Cardiac CaV1.2 channels require beta subunits for beta-adrenergic-mediated modulation but not trafficking,” J. Clin. Invest.129: 647-658 (2019).

60. Miriyala et al., “Role of CaVbeta subunits, and lack of functional reserve, in protein kinase A modulation of cardiac CaV1.2 channels,” Circ. Res. 102: e54-64 (2008).

61. Shan et al., “Phosphorylation of the ryanodine receptor mediates the cardiac fight or flight response in mice,” J. Clin. Invest. 120: 4388-4398 (2010).

62. Anwar et al., “PKC-independent signal transduction pathways increase SERCA2 expression in adult rat cardiomyocytes,” J. Mol. Cell. Cardiol. 39: 911-919 (2005).

63. Lyon et al., “Plasticity of surface structures and beta(2)-adrenergic receptor localization in failing ventricular cardiomyocytes during recovery from heart failure,” Circ. Heart Fail. 5: 357-365 (2012).

64. Basheer et al., “GJA1-20k Arranges Actin to Guide C×43 Delivery to Cardiac Intercalated Discs,” Circ. Res. 121: 1069-1080 (2017).

65. Zincarelli et al., “Analysis of AAV serotypes 1-9 mediated gene expression and tropism in mice after systemic injection,” Mol. Ther. 16: 1073-1080 (2008).

66. Chen et al., “Targeting transgene to the heart and liver with AAV9 by different promoters,” Clin. Exp. Pharmacol. Physiol. 42: 1108-1117 (2015).

67. Denegri et al., “Single delivery of an adeno-associated viral construct to transfer the CASQ2 gene to knock-in mice affected by catecholaminergic polymorphic ventricular tachycardia is able to cure the disease from birth to advanced age,” Circulation 129: 2673-2681 (2014).

68. Writing Group et al., “Heart Disease and Stroke Statistics-2016 Update: A Report from the American Heart Association,” Circulation 133: e38-360 (2016).

69. Verloop et al., “A systematic review concerning the relation between the sympathetic nervous system and heart failure with preserved left ventricular ejection fraction,” PLoS One 10: e0117332 (2015).

70. Amoasii et al., “Myotubularin and Ptdlns3P remodel the sarcoplasmic reticulum in muscle in vivo,” J. Cell Sci. 126: 1806-1819 (2013).

71. Guo and Song, AutoTT: automated detection and analysis of T-tubule architecture in cardiomyocytes,” Biophys. J. 106(12): 2729-2736 (2014).

72. Basheer et al., “Stress response protein GJA1-20k promotes mitochondrial biogenesis, metabolic quiescence, and cardioprotection against ischemia/reperfusion injury,” JCI Insight 3(20):e121900 (2018).

73. Kirchhefer et al., “Chronic beta-adrenergic stimulation reverses depressed Ca handling in mice overexpressing inhibitor-2 of protein phosphatase 1,” J. Mol. Cell. Cardiol. 125: 195-204 (2018).

74. Fu and Shaw, “CASAAV Technology to Examine Regulators of Heart Failure: Cause or Effect.” Circ. Res. 120:1846-1848 (2017).

75. Gaasch and Zile, “Left ventricular structural remodeling in health and disease: with special emphasis on volume, mass, and geometry,” J. Am. Coll. Cardiol. 58: 1733-1740 (2011).

76. Periasamy and Janssen, “Molecular basis of diastolic dysfunction,” Heart Fail. Clin. 4: 13-21 (2008).

77. Boknik et al., “Protein phosphatase activity is increased in a rat model of long-term beta-adrenergic stimulation,” Naunyn Schmiedebergs Arch. Pharmacol. 362: 222-231 (2000).

78. Valero-Munoz, et al., “Murine Models of Heart Failure with Preserved Ejection Fraction: a ‘Fishing Expedition,’” JACC Basic Transl. Sci. 2(6): 770-789 (2017).

79. Liu et al., “In Mice Subjected to Chronic Stress, Exogenous cBIN1 Preserves Calcium-Handling Machinery and Cardiac Function,” JACC Basic Transl. Sci. 5(6): 561-578 (2020).

80. Li et al., “Exogenous Cardiac Bridging Integrator 1 Benefits Mouse Hearts with Pre-existing Pressure Overload-Induced Heart Failure,” Front. Physiol. 11: 708 (2020).

1. A method for rehabilitating heart tissue or ameliorating symptoms of heart failure in a subject having experienced heart failure or under chronic stress, the method comprising, diagnosing heart failure or myocardial stress in a subject; and administering a transgene encoding a Cardiac Bridging Integrator 1 (cBIN1) to heart tissue of the subject having experienced heart failure.

2. The method of claim 1, wherein the diagnosis of heart failure or myocardial stress comprises measuring reduced cBIN1 blood levels.

3. A method for rehabilitating or increasing contractile (systolic) function or relaxation (diastolic) function in the heart of a subject having experienced heart failure, the method comprising administering a transgene encoding Cardiac Bridging Integrator 1 (cBIN1) to heart tissue of the subject, wherein after the transgene is delivered to the heart tissue and expressed, contractile function of the heart is rehabilitated or increased.

4. The method of claim 3 wherein the transgene is administered after the subject is diagnosed with heart failure.

5. The method of claim 4, wherein the diagnosis of heart failure comprises measuring reduced cBIN1 blood levels.

6. The method of claim 1, wherein the method comprises administering the transgene to myocardium.

7. The method of claim 1, wherein the transgene is administered by injection.

8. The method of claim 1, wherein the transgene comprises a vector comprising the transgene encoding cBIN1.

9. The method of claim 1, wherein the transgene comprises about 1×10¹⁰ to about 5×10¹⁰ of vector genome.

10. The method of claim 1, wherein expression of cBIN1 restructures damaged myocardium.

11. The method of claim 1, wherein expression of cBIN1 stabilizes intracellular distribution of calcium handling machinery in the myocardium.

12. The method of claim 1, wherein expression of cBIN1 reduces concentric hypertrophy in the myocardium.

13. The method of claim 1, wherein expression of cBIN1 rehabilitates or increases t-tubule microfolds or microdomains in the myocardium.

14. The method of claim 1, wherein expression of cBIN1 rehabilitates or decreases hyperphosphorylation of ryanodine receptor 2 (RyR2) in the myocardium.

15. The method of claim 1, wherein expression of cBIN1 rehabilitates or improves cardiac contractility and lusitropy.

16. The method of claim 1, wherein expression of cBIN1 rehabilitates or improves cardiac relaxation and diastolic function.

17. The method of claim 1, wherein expression of cBIN1 is prophylactic for further damage to the myocardium.

18. The method of claim 1, wherein the transgene is administered at least once.

19. The method of claim 1, wherein the subject is mammal.

20. The method of claim 1, wherein the subject is a mouse or dog.

21. The method of claim 1, wherein the subject is a human.

22. The method of claim 1, wherein the subject experiences reduced ejection fraction (HFrEF).

23. (canceled)

24. (canceled)
